(19) United States
(12) Patent Application Publication
(10) Pub. No.: US 2006/0111314 A1
(43) Pub. Date: May 25, 2006

(54) Method for treating or preventing metastasis of colorectal cancers

(75) Inventors: Yusuke Nakamura, Yokohama-shi, Kanagawa (JP); Yoichi Furukawa, Kawasaki-shi, Kanagawa (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Oncotheraphy Science, Inc., Kawasaki-shi, Kanagawa (JP); The University of Tokyo, Bunkyo-ku, Tokyo (JP)

(21) Appl. No.: 10/529,523

(22) PCT Filed: Aug. 14, 2003
(86) PCT No.: PCT/JP03/10338

§ 371 Date: Jan. 18, 2006

Related U.S. Application Data
(60) Provisional application No. 60/414,709, filed on Sep. 30, 2002.

(51) Int. Cl. A61K 48/00; C12Q 1/68; A61K 39/395

(52) U.S. Cl. 514/44; 435/6; 424/155.1

(57) ABSTRACT

Disclosed are methods of detecting metastasis of colorectal cancer using differentially expressed genes. Also disclosed are methods of identifying agents for treating colorectal cancer or preventing metastasis of colorectal cancer.

The present application is related to U.S. Ser. No. 60/414,709, filed Sep. 30, 2002, which is incorporated herein by reference.

### TECHNICAL FIELD

The invention relates to methods of treating colorectal cancers and preventing metastasis of colorectal cancers.

### BACKGROUND ART

Liver metastasis is a major cause of death among patients with colorectal cancer (CRC). Despite progress that has been achieved with therapeutic approaches, a complete cure awaits more effecting strategies. Prevention or effective treatment of liver metastasis will save the lives of thousands of patients.

The process of metastasis involves multiple steps that include release of cancer cells from the primary site, intravasation to neighboring vessels, transport to the site of metastasis through blood flow, extravasation and/or infarction to the distant organ, and re-growth of the invading cells with acquisition of nutrition in the new environment. Therefore multiple genes are expected to be associated with the process of metastasis. Although many investigators have been working on this clinically important issue, the precise mechanisms or identification of the critical genes remain to be clarified. A number of molecules associated with liver metastasis have been reported, but as most studies have focused on only one or a few molecules, the importance of each genes in the complex process remains obscure.

Due to the progress in microarray technology, expression levels of thousands of genes can be identified in a single experiment and classification of cancer based on altered expression of multiple genes in tumor tissues is suggested (Golub et al., Science 286: 531-7 (1999); Alizadeh et al., Nature 403: 503-11 (2000)). cDNA microarray technologies have enabled to obtain comprehensive profiles of gene expression in normal and malignant cells, and compare the gene expression in malignant and corresponding normal cells (Okabe et al., Cancer Res 61:2129-37 (2001); Kitahara et al., Cancer Res 61: 3544-9 (2001); Lin et al., Oncogene 21:4120-8 (2002); Hasegawa et al., Cancer Res 62:7012-7 (2002)). This approach enables to disclose the complex nature of cancer cells, and helps to understand the mechanism of carcinogenesis as well as metastasis of cancer. Identification of genes that are deregulated in tumors can lead to more precise and accurate diagnosis of individual cancers, and to develop novel therapeutic targets (Bienz and Clevers, Cell 103:311-20 (2000)).

Recently two groups detected genes responsible for metastasis of malignant melanomas, using cDNA microarrays. One group compared the expression profiles of highly metastatic melanoma cells with less metastatic cells established from the same cell lines (Clark et al., Nature 406: 532-5 (2000)). On the other hand, the other group analyzed expression profiles among various melanoma cell lines and primary melanomas (Bittner et al., Nature 406: 536-40 (2000)). Furthermore, to disclose the mechanisms underling liver metastasis of colorectal cancer, the present inventors previously analyzed expression profiles of 10 primary tumors and their corresponding metastatic lesions using a cDNA microarray containing 9121 genes (Yanagawa et al., Neoplasia 3: 395-401 (2001)).

Studies designed to reveal mechanisms of carcinogenesis have already facilitated identification of molecular targets for anti-tumor agents. Various agents designed to suppress oncogenic activity of specific gene products have been revealed to be effective for treating tumors (He et al., Cell 99:335-45 (1999); Lin et al., Cancer Res 61:6345-9 (2001); Fujita et al., Cancer Res 61:7722-6 (2001)). Therefore, gene products commonly up-regulated in cancerous cells may serve as potential targets for developing novel anti-cancer agents.

CD8+ cytotoxic T lymphocytes (CTLs) have been demonstrated to recognize epitope peptides derived from tumor-associated antigens (TAAs) presented on MHC Class I molecule, and lyse tumor cells. Since the discovery of MAGE family as the first example of TAAs, many other TAAs have been discovered using immunological approaches (Boon, Int J Cancer 54: 177-80 (1993); Boon and van der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994)). Some of the discovered TAAs are now at the stage of clinical development as targets of immunotherapy. TAAs discovered so far include MAGE (van der Bruggen et al., Science 254: 1643-7 (1991)), gp100 (Kawakami et al., J Exp Med 180: 347-52 (1994)), SART (Shichijo et al., J Exp Med 187: 277-88 (1998)) and NY-ESO-1 (Chen et al., Proc Natl Acad Sci USA 94: 1914-8 (1997)). On the other hand, gene products which had been demonstrated to be specifically overexpressed in tumor cells, have been shown to be recognized as targets inducing cellular immune responses. Such gene products include p53 (Umano et al., Brit J Cancer 84: 1052-7 (2001)), HER2/neu (Tanaka et al., Brit J Cancer 84: 94-9 (2001)), CEA (Nukaya et al., Int J Cancer 80: 92-7 (1999)) and so on.

In spite of significant progress in basic and clinical research concerning TAAs (Rosenbeg et al., Nature Med 4: 321-7 (1998); Mukherji et al., Proc Natl Acad Sci USA 92: 8078-82 (1995); Hu et al., Cancer Res 56: 2479-83 (1996)), only limited number of candidate TAAs for the treatment of adenocarcinomas, including colorectal cancer, are available. TAAs abundantly expressed in cancer cells, and at the same time which expression is restricted to cancer cells would be promising candidates as immunotherapeutic targets. Further, identification of new TAAs inducing potent and specific antitumor immune responses is expected to encourage clinical use of peptide vaccination strategy in various types of cancer (Boon and can der Bruggen, J Exp Med 183: 725-9 (1996); van der Bruggen et al., Science 254: 1643-7 (1991); Brichard et al., J Exp Med 178: 489-95 (1993); Kawakami et al., J Exp Med 180: 347-52 (1994); Shichijo et al., J Exp Med 187: 277-88 (1998); Chen et al., Proc Natl Acad Sci USA 94: 1914-8 (1997); Harris, J Natl Cancer Inst 88: 1442-5 (1996); Butterfield et al., Cancer Res 59: 3134-42 (1999); Vissers et al., Cancer Res 59: 5554-9 (1999); van der Burg et al., J Immunol 156: 3308-14 (1996); Tanaka et al., Cancer Res 57: 4465-8 (1997); Fujie et al., Int J Cancer 80: 169-72 (1999); Kikuchi et al., Int J Cancer 81: 459-66 (1999); Oiso et al., Int J Cancer 81: 387-94 (1999)).

### SUMMARY OF THE INVENTION

The present invention is based on the discovery of a pattern of gene expression correlated with metastasis of colorectal cancer.

To disclose the mechanism of liver metastasis of colorectal cancer and identify novel diagnostic markers and/or drug targets for the treatment and prevention of metastasis, the present inventors analyzed the expression profiles of fifteen primary colorectal cancers (CRCs) with liver metastasis and non-cancerous colonic mucosae using a genome-wide cDNA microarray containing 23040 human genes. As a result, a number of genes whose expression was frequently enhanced in the primary lesions compared to their corresponding non-cancerous mucosae were identified. Among these genes, comparison of the data with the expression profiles of 11 colon cancer tissues without liver metastasis and 9 premalignant tumors of the colon identified 163 genes whose expression was elevated in tumors with metastasis but not in those without metastasis. These genes are collectively referred to herein as “MLX nucleic acids”, “MLX polynucleotides” or “MLX genes” and the corresponding encoded polypeptides are referred to as “MLX polypeptides” or “MLX proteins”.

Accordingly, the invention features a method of diagnosing or determining a predisposition to metastatic lesions of colorectal cancer in a subject by determining a level of expression of metastasis-associated gene in a patient derived biological sample. By metastasis-associated gene is meant a gene that is characterized by a level of expression which differs in a cell obtained from a primary colorectal cancer cell with metastasis compared to a normal cell or a primary colorectal tumor without metastasis. A normal cell is one obtained from colorectal tissue or benign adenomas. A metastasis-associated gene includes for example MLXs 1-163 (Table 1). An increase of the level of expression of the gene compared to a normal control level of the gene indicates that the subject suffers from or is at risk of developing metastatic lesions of colorectal cancer.

By normal control level is meant a level of gene expression detected in a normal, healthy individual or in a population of individuals known not to be suffering from metastatic lesions of colorectal cancer. A control level is a single expression pattern derived from a single reference population or from a plurality of expression patterns. For example, the control level can be a database of expression patterns from previously tested cells.

Alternatively, expression of a panel of metastasis-associated genes in the sample is compared to a metastatic control level of the same panel of genes. By metastatic control level is meant the expression profile of the metastasis-associated genes found in a population suffering from metastatic lesions of colorectal cancer.

Gene expression is increased 10%, 25% or 50% compared to the control level. Alternately, gene expression is increased 1, 2, 5 or more fold compared to the control level. Expression is determined by detecting, for example, by hybridization (e.g., on a chip) of metastasis-associated gene probe to a gene transcript (e.g., mRNA) of the patient-derived tissue sample.

The patient derived biological sample is any biological sample from a test subject, e.g., a patient known to or suspected of having metastatic lesions of colorectal cancer. For example, the biological sample contains tissues from the test subject encompassing a primary colorectal cancer cell or a metastatic colorectal cancer cell. A metastatic cell is a cancer cell which has migrated from a primary tumor site to a secondary tumor site.

The invention also provides metastatic reference expression profile of a gene expression level of two or more of MLXs 1-163.

Further, a method of screening for a compound for treating colorectal cancer or preventing metastasis of colorectal cancer is provided. The method includes contacting an MLX polypeptide with a test compound, and selecting the test compound that bind to the MLX polypeptide.

Furthermore, the present invention provides a method of screening for a compound for treating colorectal cancer or preventing metastasis of colorectal cancer, wherein the method includes contacting a MLX polypeptide with a test compound, and selecting a compound that suppresses the biological activity of the MLX polypeptide.

The present invention further provides a method of screening for a compound for treating colorectal cancer or preventing metastasis of colorectal cancer, wherein the method includes contacting a cell expressing one or more of the MLX polypeptides with a test compound, and selecting the test compound that suppresses the expression level of one or more MLX polypeptides.

Furthermore, the present invention provides a method of screening for a compound for treating colorectal cancer or preventing metastasis of colorectal cancer, wherein the method includes contacting a test compound and a vector comprising a reporter gene downstream of a transcriptional regulatory region of MLX genes under a suitable condition for the expression of the reporter gene, and selecting the test compound that inhibits the expression of the reporter gene.

The present application also provides a composition for treating colorectal cancer or preventing metastasis of colorectal cancer. The composition may be, for example, an anti-cancer agent. The composition can be described as at least a portion of the antisense S-oligonucleotides or small interfering RNA (siRNA) of the MLX polynucleotides or antibody or fragment of the antibody against the MLX proteins. The compositions may be also those comprising the compounds selected by the present methods of screening for compounds for treating colorectal cancer or preventing metastasis of colorectal cancer.

The course of action of the pharmaceutical composition is desirably to inhibit development of metastasis of colorectal cancer or inhibit growth or proliferation of the primary lesion of malignant colorectal cancer. The pharmaceutical composition may be applied to mammals including humans and domesticated mammals.

Furthermore, the present invention provides a composition for treating colorectal cancer or preventing metastasis of colorectal cancer comprising an MLX protein, a polynucleotide encoding the protein or a vector comprising the polynucleotide. Such compositions are expected to induce anti-tumor immunity.

The present invention further provides methods for treating colorectal cancer or preventing metastasis of colorectal cancer using any of the compositions provided by the present invention.

The invention also provides a kit with a detection reagent which binds to one or more MLX nucleic acid sequences or which binds to a gene product encoded by the nucleic acid sequences. Also provided is an array of nucleic acids that binds to one or more MLX nucleic acids. Such kits and arrays are expected to be useful for diagnosing metastasis of colorectal cancer.

It is to be understood that both the foregoing summary of the invention and the following detailed description are of a preferred embodiment, and not restrictive of the invention or other alternate embodiments of the invention.

### DETAILED DESCRIPTION OF THE INVENTION

The words “a”, “an” and “the” as used herein mean “at least one” unless otherwise specifically indicated.

The present invention is based in part on the discovery of changes (increase) in expression patterns of multiple nucleic acid sequences in primary cancer tissue from patients with colorectal cancer with liver metastasis. The differences in gene expression were identified using laser-capture microdissection (LCM) and a comprehensive cDNA microarray system. The differentially expressed genes identified herein are used for diagnostic purposes and to develop gene targeted therapeutic approaches to treat colorectal cancer, especially malignant colorectal cancer with metastasis, and to inhibit metastasis of colorectal cancer.

The genes whose expression levels are increased in patients with metastatic lesions of colorectal cancer are summarized in Table 1 and are collectively referred to herein as “metastasis-associated genes”, “MLX nucleic acids” or “MLX polynucleotides” and the corresponding encoded polypeptides are referred to as “MLX polypeptides” or “MLX proteins”. Unless indicated otherwise, “MLX” is meant to refer to any of the sequences disclosed herein (e.g., MLX 1-163). The genes have been previously described and are presented along with a database accession number.

By measuring expression of the various genes or activity of protein encoded by the genes in a biological sample, metastasis of colorectal cancer can be determined. Similarly, by measuring the expression of these genes or activity of protein encoded by the genes in response to various agents, agents for treating colorectal cancer or preventing metastasis of colorectal cancer can be identified.

Diagnosing Metastasis of Colorectal Cancer

The present invention provides a method of diagnosing a predisposition to developing metastatic lesions of colorectal cancer in a subject. Specifically, the method comprises determining a level of expression of metastasis-associated gene in a patient derived biological sample. An increase in the expression level of metastasis-associated gene (e.g., MLX 1-163) compared to a normal control level of the metastasis-associated gene indicates that the patient suffers from or is at risk of developing metastatic lesions of colorectal cancer.

The invention involves determining (e.g., measuring) the expression of at least one, and up to all the MLX sequences listed in Table 1. To confirm the diagnosis result obtained with one gene or for obtaining a more reliable diagnosis result, one can determine the expression level of a plurality of MLX genes according to the present method. The expression of 1, 2, 3, 4, 5, 25, 35, 50, or 100 or more of the sequences represented by MLXs 1-163 is determined and if desired, expression of these sequences can be determined along with other sequences whose level of expression is known to be altered according to one of the herein described parameters or conditions, e.g., metastatic lesions of colorectal cancer or non-metastatic lesions of colorectal cancer.

Using sequence information provided by the GenBank database entries for the known sequences, the metastasis-associated genes are detected and measured using techniques well known to one of ordinary skill in the art. For example, sequences within the sequence database entries corresponding to MLX sequences, can be used to construct probes for detecting MLX RNA sequences in hybridization, e.g., northern blot hybridization analyses. As another example, the sequences can be used to construct primers for specifically amplifying the MLX sequences in, e.g., amplification-based detection methods such as reverse-transcription based polymerase chain reaction (RT-PCR). According to the method of the present invention, gene transcript including MLX RNA such as mRNA in biological sample can be detected. Furthermore, the detection of MLX sequences can be conducted using a DNA chip. Specifically, one or more nucleotides hybridizing with the MLX RNAs can be immobilized on a DNA chip for the detection.

The expression level of metastasis-associated gene is determined in the diagnosis of the present invention by (1) detecting mRNA of the metastasis-associated gene, (2) detecting protein encoded by the metastasis-associated gene, or (3) detecting the biological activity of the protein encoded by the metastasis-associated gene.

Detection methods for mRNA are known to those skilled in the art. For example, the levels of mRNAs corresponding to the metastasis-associated gene can be estimated by Northern blotting or RT-PCR. The nucleotide sequences of the genes depicted in Table 1 can be used to design the nucleotide sequences for probes or primers to quantify the gene according to conventional methods.

Also the expression level of the metastasis-associated gene can be analyzed based on activity or quantity of protein encoded by the gene (e.g., MLX 1-163; Table 1). A method for determining the quantity of the metastasis-associated protein is shown in below. For example, immunoassay method is useful for determination of the protein in biological material. Any suitable method can be selected for the determination of the activity of the protein encoded by the metastasis-associated gene according to the activity of each protein to be analyzed.

Expression level of one or more of the metastasis-associated gene in biological samples, e.g., a patient derived tissues sample is then compared to the expression levels of the same sequences in a reference sample. Any biological materials derived from a subject can be used for the determination of the expression level of metastasis-associated gene including one or more cells, such as primary colorectal cancer tissue cell obtained by biopsy (i.e., patient derived tissue sample). Gene expression is also measured from blood or other bodily fluids such as sputum. Thus, the “biological sample” of the present invention includes a bodily tissue or a bodily fluid, e.g., biological fluid (such as blood, serum or sputum) and includes cells purified from a tissue. The test biological sample includes tissues and cell samples from a subject known to contain, or to be suspected of containing, metastatic lesions of colorectal cancer cells. Preferably, the test biological sample comprises a colorectal cancer cell.

Reference biological samples (control) are, for example, derived from a tissue type similar to the test biological sample. The reference biological samples include, for example, one or more cells for which the compared parameter is known, i.e., cancerous, non-cancerous, metastatic or non-metastatic. Alternatively, the control biological sample is derived from a database of molecular information derived from cells for which the assayed parameter or condition is known. A control level may be a single expression pattern derived from a single reference population or from a plurality of expression patterns. By normal control level is meant the expression profile of the metastasis-associated genes typically found in a sample derived from tissue or patient not suffering from metastatic lesions of colorectal cancer. An increase in expression of MLX 1-163 in the test biological sample compared to the normal control level indicates that the subject is suffering from or is at risk of developing metastatic lesions of colorectal cancer.

The subject is preferably a mammal. The mammal can be, e.g., a human, non-human primate, mouse, rat, dog, cat, horse or cow.

Whether or not the gene expression levels in the biological sample compared to the reference sample reveals the presence of the measured parameter depends on the composition of the reference sample. For example, if the reference sample is a cell population composed of non-metastatic cells, a similar gene expression level in the test biological sample consisting of a cell population and reference cell population indicates the test biological sample is non-metastatic. Conversely, if the reference sample is a cell population made up of metastatic cells, a similar gene expression profile between the biological sample and the reference sample indicates that the test biological sample consisting of a cell population includes metastatic cells.

An MLX sequence in a biological sample can be considered increased in levels of expression if its expression level is higher from the reference sample by more than 1.0, 1.5, 2.0, 5.0, 10.0 or more fold from the expression level of the corresponding MLX sequence in the reference sample. According to the present method, when one or more of the metastatic lesions of colorectal cancer-associated genes are increased at least 10% (e.g., 50%, 60%, 70%, 80%, 90% or more) than the normal control level, the subject is diagnosed to suffer from or is at risk of developing metastatic lesions of colorectal cancer.

If desired, comparison of differentially expressed sequences between a biological sample and a reference sample can be done with respect to a control nucleic acid whose expression is independent of the parameter or condition being measured. For example, a control nucleic acid is one which is known not to differ depending on the cancerous or non-cancerous state, or metastatic or non-metastatic state of the cell. Expression levels of the control nucleic acid in the test and reference nucleic acid can be used to normalize signal levels in the compared samples. Control genes can be, e.g., β-actin, glyceraldehyde 3-phosphate dehydrogenase or ribosomal protein P1.

The test biological sample may be compared to multiple reference biological samples. Each of the multiple reference biological samples may differ in the known parameter. Thus, a test biological sample consisting of a population of cells may be compared to a second reference biological sample known to contain, e.g., metastatic lesions of colorectal cancer cells, as well as a second reference population known to contain, e.g., non-metastatic lesions of colorectal cancer cells (normal cells).

When alterations in gene expression are associated with gene amplification or deletion, sequence comparisons in test and reference samples can be made by comparing relative amounts of the examined DNA sequences in the test and reference samples.

The differentially expressed MLX sequences identified herein also allow for the course of treatment of colorectal cancer to be monitored. In this method, a test biological sample is provided from a subject undergoing treatment for colorectal cancer. If desired, biological samples are obtained from the subject at various time points before, during or after treatment. Expression of one or more of the MLX sequences, in the cell population is then determined and compared to a reference biological sample whose metastatic lesions of colorectal cancer state is known. The reference biological sample is obtained from a tissue or subject that has not been exposed to the treatment.

If the reference biological sample contains non-metastatic colorectal cancer cells, a similarity in expression between MLX sequences in the test biological sample and the reference biological sample indicates that the treatment is efficacious. However, a difference in expression between MLX sequences in the test biological sample and this reference biological sample indicates a less favorable clinical outcome or prognosis.

By “efficacious” is meant that the treatment leads to a reduction in expression of a pathologically up-regulated gene, increase in expression of a pathologically down-regulated gene or a decrease in size, prevalence or metastatic potential of colorectal cancer in a subject. When treatment is applied prophylactically, “efficacious” means that the treatment retards or prevents metastatic lesions of colorectal cancer from forming, e.g., detection of secondary tumors in an anatomical site which differs from that of the primary tumor. Assessment of metastatic lesions of colorectal cancer is made using standard clinical protocols.

Efficaciousness is determined in association with any known method for diagnosing or treating colorectal cancer and metastatic lesions of colorectal cancer. Colorectal cancer is diagnosed for example, by rectal examination, colonoscopy, barium enema, blood test, e.g., for anemia or CEA antigen.

Also provided is a method of assessing the prognosis of a subject with malignant colorectal cancer by comparing the expression of one or more MLX sequences in a test biological sample to the expression of the sequences in a reference biological sample derived from patients over a spectrum of disease stages. By comparing gene expression of one or more MLX sequences in the biological sample and the reference biological sample(s), or by comparing the pattern of gene expression over time in biological samples derived from the subject, the prognosis of the subject can be assessed.

An increase in expression of one or more of the sequences MLXs 1-163 compared to a normal control indicates less favorable prognosis.

Primary Colorectal Cancer Reference Expression Profile

A primary colorectal cancer reference expression profile is provided by the present invention. Such expression profiles of the present invention comprise a pattern of gene expression of two or more MLX genes of primary colorectal cancer cells with metastasis, non-metastatic colorectal cancer cells or normal cells. The expression profile can be used in diagnosing a predisposition to developing metastatic lesions of colorectal cancer in a subject, monitoring the course of treatment of colorectal cancer and assessing prognosis of a subject with malignant colorectal cancer.

Screening Compounds for Treating Colorectal Cancer or Preventing Metastasis of Colorectal Cancer

The present invention provides a method of screening for a compound for treating colorectal cancer or preventing metastasis of colorectal cancer using one or more MLX polypeptides. An embodiment of this screening method comprises the steps of: (a) contacting a test compound with an MLX polypeptide, (b) detecting the binding activity between the polypeptide and the test compound, and (c) selecting a compound that binds to the MLX polypeptide.

In another embodiment of the method for screening a compound for treating colorectal cancer or preventing metastasis of colorectal cancer of the present invention, the method utilizes the biological activity of the MLX polypeptide as an index. This screening method includes the steps of: (a) contacting a test compound with the MLX polypeptide; (b) detecting the biological activity of the MLX polypeptide of step (a); and (c) selecting a compound that suppresses the biological activity of the MLX polypeptide in comparison with the biological activity detected in the absence of the test compound.

The MLX polypeptide of the present invention used for the screening are selected from following polypeptides:

(1) a polypeptide comprising the amino acid sequence encoded by a polynucleotide selected from the group consisting of MLXs 1-163;
(2) a polypeptide that comprises the amino acid sequence encoded by a polynucleotide selected from the group consisting of MLXs 1-163, in which one or more amino acids are substituted, deleted, inserted, and/or added and that has a biological activity equivalent to a protein consisting of the amino acid sequence encoded by the polynucleotide; and
(3) a polypeptide encoded by a polynucleotide that hybridizes under stringent conditions to a polynucleotide selected from the group consisting of MLXs 1-163, wherein the polypeptide has a biological activity equivalent to a polypeptide consisting of the amino acid sequence encoded by the polynucleotide selected from the group consisting of MLXs 1-163.

In the present invention, the term “biological activity” refers to activities such as occurrence of metastasis including the activity to develop metastatic legions, i.e., generate a metastatic lesion (onset of metastasis), promote metastasis and growth or proliferation of metastatic lesion. The activity to generate a metastatic lesion and promote metastasis includes release of cancer cells from the primary site, intravasation to neighboring vessels, transport to the site of metastasis through blood flow, and extravasation and/or infarction to the distant organ. Whether the subject polypeptide has the biological activity or not can be judged by introducing the DNA encoding the subject polypeptide into a cell expressing the respective polypeptide, and detecting occurrence or promotion of metastasis, growth or proliferation of the cells, increase in colony forming activity, etc.

Methods for preparing polypeptides having the biological activity of a given protein are well known by a person skilled in the art and include known methods of introducing mutations into the protein. For example, one skilled in the art can prepare polypeptides having the biological activity of the human MLX protein by introducing an appropriate mutation in the amino acid sequence of either of these proteins by site-directed mutagenesis (Hashimoto-Gotoh et al., Gene 152:271-5 (1995); Zoller and Smith, Methods Enzymol 100: 468-500 (1983); Kramer et al., Nucleic Acids Res. 12:9441-9456 (1984); Kramer and Fritz, Methods Enzymol 154: 350-67 (1987); Kunkel, Proc Natl Acad Sci USA 82: 488-92 (1985); Kunkel, Methods Enzymol 85: 2763-6 (1988)). Amino acid mutations can occur in nature, too. The MLX polypeptide includes those proteins having the amino acid sequences of the human MLX protein in which one or more amino acids are mutated, provided the resulting mutated polypeptides have the biological activity of the human MLX protein. The number of amino acids to be mutated in such a mutant is generally 10 amino acids or less, preferably 6 amino acids or less, and more preferably 3 amino acids or less.

Mutated or modified proteins, proteins having amino acid sequences modified by substituting, deleting, inserting, and/or adding one or more amino acid residues of a certain amino acid sequence, have been known to retain the original biological activity (Mark et al., Proc Natl Acad Sci USA 81: 5662-6 (1984); Zoller and Smith, Nucleic Acids Res 10:6487-500 (1982); Dalbadie-McFarland et al., Proc Natl Acad Sci USA 79: 6409-13 (1982)).

The amino acid residue to be mutated is preferably mutated into a different amino acid in which the properties of the amino acid side-chain are conserved (a process known as conservative amino acid substitution). Examples of properties of amino acid side chains are hydrophobic amino acids (A, I, L, M, F, P, W, Y, V), hydrophilic amino acids (R, D, N, C, E, Q, G, H, K, S, T), and side chains having the following functional groups or characteristics in common: an aliphatic side-chain (G, A, V, L, I, P); a hydroxyl group containing side-chain (S, T, Y); a sulfur atom containing side-chain (C, M); a carboxylic acid and amide containing side-chain (D, N, E, Q); a base containing side-chain (R, K, H); and an aromatic containing side-chain (H, F, Y, W). Note, the parenthetic letters indicate the one-letter codes of amino acids.

An example of a polypeptide to which one or more amino acids residues are added to the amino acid sequence of human MLX protein is a fusion protein containing the human MLX protein. Fusion proteins are, fusions of the human MLX protein and other peptides or proteins, and are included in the MLX protein described herein. Fusion proteins can be made by techniques well known to a person skilled in the art, such as by linking the DNA encoding the human MLX protein with DNA encoding other peptides or proteins, so that the frames match, inserting the fusion DNA into an expression vector and expressing it in a host. There is no restriction as to the peptides or proteins fused to the MLX protein.

Known peptides that can be used as peptides that are fused to the MLX protein include, for example, FLAG (Hopp et al., Biotechnology 6: 1204-10 (1988)), 6×His containing six His (histidine) residues, 10×His, Influenza agglutinin (HA), human c-myc fragment, VSP-GP fragment, p18HIV fragment, T7-tag, HSV-tag, E-tag, SV40T antigen fragment, Ick tag, α-tubulin fragment, B-tag, Protein C fragment and the like. Examples of proteins that may be fused to an MLX protein include GST (glutathione-S-transferase), Influenza agglutinin (HA), immunoglobulin constant region, β-galactosidase, MBP (maltose-binding protein) and such.

Fusion proteins can be prepared by fusing commercially available DNA, encoding the fusion peptides or proteins discussed above, with the DNA encoding the MLX polypeptide and expressing the fused DNA prepared. A commercially available epitope-antibody system can be used (Experimental Medicine 13: 85-90 (1995)) for expressing such fusion proteins. Vectors which can express a fusion protein with, for example, β-galactosidase, maltose binding protein, glutathione S-transferase, green florescence protein (GFP) and so on by the use of its multiple cloning sites are commercially available.

An alternative method known in the art to isolate polypeptides having the biological activity of any of the MLX proteins is, for example, the method using a hybridization technique (Sambrook et al., Molecular Cloning 2nd ed. 9.47-9.58, Cold Spring Harbor Lab. Press (1989)). One skilled in the art can readily isolate a DNA having high homology with a whole or part of the DNA sequence encoding the human MLX protein, and isolate polypeptides having the biological activity of the human MLX protein from the isolated DNA. The MLX polypeptides include those that are encoded by DNA that hybridize with a whole or part of the DNA sequence encoding the human MLX protein and have the biological activity of the human MLX protein. These polypeptides include mammal homologues corresponding to the protein derived from human (for example, a polypeptide encoded by a monkey, rat, rabbit and bovine gene). In isolating a cDNA highly homologous to the DNA encoding the human MLX protein from animals, it is particularly preferable to use tissues from colorectal cancers with metastasis.

The condition of hybridization for isolating a DNA encoding a polypeptide having the biological activity of the human MLX protein can be routinely selected by a person skilled in the art. For example, hybridization may be performed by conducting prehybridization at 68° C. for 30 min or longer using “Rapid-hyb buffer” (Amersham LIFE SCIENCE), adding a labeled probe, and warming at 68° C. for 1 hour or longer. The following washing step can be conducted, for example, in a low stringent condition. A low stringent condition is, for example, 42° C., 2×SSC, 0.1% SDS, or preferably 50° C., 2×SSC, 0.1% SDS. More preferably, high stringent conditions are used. A high stringent condition is, for example, washing 3 times in 2×SSC, 0.01% SDS at room temperature for 20 min, then washing 3 times in 1×SSC, 0.1% SDS at 37° C. for 20 min, and washing twice in 1×SSC, 0.1% SDS at 50° C. for 20 min. However, several factors, such as temperature and salt concentration, can influence the stringency of hybridization and one skilled in the art can suitably select the factors to achieve the requisite stringency.

In place of hybridization, a gene amplification method, for example, the polymerase chain reaction (PCR) method, can be utilized to isolate a DNA encoding a polypeptide having the biological activity of the human MLX protein, using a primer synthesized based on the sequence information of the protein encoding DNA.

Polypeptides that have the biological activity of the human MLX protein encoded by the DNA isolated through the above hybridization techniques or gene amplification techniques, normally have a high homology to the amino acid sequence of the human MLX protein. “High homology” typically refers to a homology of 40% or higher, preferably 60% or higher, more preferably 80% or higher, even more preferably 95% or higher. The homology of a polypeptide can be determined by following the algorithm in “Wilbur and Lipman, Proc Natl Acad Sci USA 80: 726-30 (1983)”.

An MLX polypeptide used in the method of the present invention may have variations in amino acid sequence, molecular weight, isoelectric point, the presence or absence of sugar chains, or form, depending on the cell or host used to produce it or the purification method utilized. Nevertheless, so long as it has a biological activity equivalent to that of the human MLX protein, it may be used in the method of the present invention and such methods utilizing polypeptides with a biological activity equivalent to the MXL protein are within the scope of the present invention.

The MLX polypeptides used in the present invention can be prepared as recombinant proteins or natural proteins, by methods well known to those skilled in the art. A recombinant protein can be prepared by inserting a DNA, which encodes the MLX polypeptide, into an appropriate expression vector, introducing the vector into an appropriate host cell, obtaining the extract, and purifying the polypeptide.

Specifically, when E. coli is used as a host cell to prepare an MLX polypeptide, the vector should have “ori” to be amplified in E. coli and a marker gene for selecting transformed E. coli (e.g., a drug-resistance gene selected by a dmg such as ampicillin, tetracycline, kanamycin, chloramphenicol or the like). In addition, the expression vector to be expressed in E. coli should have a promoter, for example, lacZ promoter (Ward et al., Nature 341: 544-6 (1989); FASEB J 6: 2422-7 (1992)), araB promoter (Better et al., Science 240: 1041-3 (1988)), or T7 promoter or the like, that can efficiently express the desired gene in E. coli. In that respect, pGEX-5X-1 (Pharmacia), “QIAexpress system” (Qiagen), pEGFP and pET (in this case, the host is preferably BL21 which expresses T7 RNA polymerase), for example, can be used instead of the above vectors. Additionally, the vector may also contain a signal sequence for polypeptide secretion. An exemplary signal sequence that directs the polypeptide to be secreted to the periplasm of the E. coli is the peIB signal sequence (Lei et al., J Bacteriol 169: 4379 (1987)). Means for introducing the vectors into the target host cells include, for example, the calcium chloride method and the electroporation method.

In addition to E. coli, for example, expression vectors derived from mammals (for example, pcDNA3 (Invitrogen) and pEGF-BOS (Nucleic Acids Res 18(17): 5322 (1990)), pEF, pCDM8), expression vectors derived from insect cells (for example, “Bac-to-BAC baculovirus expression system” (GIBCO BRL), pBacPAK8), expression vectors derived from plants (e.g., pMH1, pMH2), expression vectors derived from animal viruses (e.g., pHSV, pMV, pAdexLcw), expression vectors derived from retroviruses (e.g., pZIpneo), expression vector derived from yeast (e.g., “Pichia Expression Kit” (Invitrogen), pNV11, SP-Q01) and expression vectors derived from Bacillus subtlis (e.g., pPL608, pKTH50) can be used for producing the MLX polypeptide.

In order to express the vector in animal cells, such as CHO, COS or NIH3T3 cells, the vector should have a promoter necessary for the expression in such cells, for example, the SV40 early promoter (Rigby in Williamson (ed.), Genetic Engineering, vol. 3. Academic Press, London, 83-141 (1982)), the MMLV-LTR promoter, the EFla promoter (Mizushima et al., Nucleic Acids Res 18: 5322 (1990); Kim et al., Gene 91: 217-23 (1990)), the CAG promoter (Niwa et al., Gene 108: 193-200 (1991)), the RSV LTR promoter (Cullen, Methods in Enzymology 152: 684-704 (1987)), the SRα promoter (Takebe et al., Mol Cell Biol 8: 466 (1988)), the CMV immediate early promoter (Seed and Aruffo, Proc Natl Acad Sci USA 84: 3365-9 (1987)), the SV40 late promoter (Gheysen and Fiers, J Mol Appl Genet 1: 385-94 (1982)), the Adenovirus late promoter (Kaufman et al., Mol Cell Biol 9: 946 (1989)), the HSV TK promoter and the like, and preferably a marker gene for selecting transformants (for example, a drug resistance gene selected by a drug (e.g., neomycin, G418)). Examples of known vectors with these characteristics include, for example, pMAM, pDR2, pBK-RSV, pBK-CMV, pOPRSV and pOP13. The introduction of the gene into animal cells to express a foreign gene can be performed according to any methods, for example, the electroporation method (Chu et al., Nucleic Acids Res 15: 1311-26 (1987)), the calcium phosphate method (Chen and Okayama, Mol Cell Biol 7: 2745-52 (1987)), the DEAE dextran method (Lopata et al., Nucleic Acids Res 12: 5707-17 (1984); Sussman and Milman, Mol Cell Biol 4: 1642-3 (1985)), the Lipofectin method (Derijard, B Cell 7: 1025-37 (1994); Lamb et al., Nature Genetics 5: 22-30 (1993): Rabindran et al., Science 259: 230-4 (1993)) and so on.

In addition, methods may be used to express a gene stably and, at the same time, to amplify the copy number of the gene in cells. For example, a vector comprising the complementary DHFR gene (e.g., pCHO I) may be introduced into CHO cells in which the nucleic acid synthesizing pathway is deleted, and then amplified by methotrexate (MTX). Furthermore, in case of transient expression of a gene, the method wherein a vector comprising a replication origin of SV40 (pcD, etc.) is transformed into COS cells comprising the SV40 T antigen expressing gene on the chromosome can be used.

An MLX polypeptide obtained as above may be isolated from inside or outside (such as medium) of host cells, and purified as a substantially pure homogeneous polypeptide. The term “substantially pure” as used herein in reference to a given polypeptide means that the polypeptide is substantially free from other biological macromolecules. The substantially pure polypeptide is at least 75% (e.g., at least 80, 85, 95 or 99%) pure by dry weight. Purity can be measured by any appropriate standard method, for example by column chromatography, polyacrylamide gel electrophoresis or HPLC analysis. The method for polypeptide isolation and purification is not limited to any specific method; in fact, any standard method may be used.

For instance, column chromatography, filter, ultrafiltration, salt precipitation, solvent precipitation, solvent extraction, distillation, immunoprecipitation, SDS-polyacrylamide gel electrophoresis, isoelectric point electrophoresis, dialysis and recrystallization may be appropriately selected and combined to isolate and purify the polypeptide.

Examples of chromatography include, for example, affinity chromatography, ion-exchange chromatography, hydrophobic chromatography, gel filtration, reverse phase chromatography, adsorption chromatography and such (Strategies for Protein Purification and Characterization: A Laboratory Course Manual. Ed. Daniel R. Marshak et al., Cold Spring Harbor Laboratory Press (1996)). These chromatographies may be performed by liquid chromatography, such as HPLC and FPLC.

Also when the MLX polypeptide is expressed within host cells (for example, animal cells and E. coli) as a fusion protein with glutathione-S-transferase protein or as a recombinant protein supplemented with multiple histidines, the expressed recombinant protein can be purified using a glutathione column or nickel column. Alternatively, when the MLX polypeptide is expressed as a protein tagged with c-myc, multiple histidines or FLAG, it can be detected and purified using antibodies to c-myc, His or FLAG, respectively.

After purifying the fusion protein, it is also possible to exclude regions other than the objective polypeptide by cutting with thrombin or factor-Xa as required.

A natural protein can be isolated by methods known to a person skilled in the art, for example, by contacting the affinity column, in which antibodies binding to the MLX protein described below are bound, with the extract of tissues or cells expressing the MLX polypeptide. The antibodies can be polyclonal antibodies or monoclonal antibodies.

The MLX polypeptide to be contacted with a test compound can be, for example, a purified polypeptide, a soluble protein, a form bound to a carrier or a fusion protein fused with other polypeptides. Examples of supports that may be used for binding proteins include insoluble polysaccharides, such as agarose, cellulose and dextran; and synthetic resins, such as polyacrylamide, polystyrene and silicon; preferably commercial available beads and plates (e.g., multi-well plates, biosensor chip, etc.) prepared from the above materials may be used. When using beads, they may be filled into a column.

The binding of a protein to a support may be conducted according to routine methods, such as chemical bonding and physical adsorption. Alternatively, a protein may be bound to a support via antibodies that specifically recognizing the protein. Moreover, binding of a protein to a support can be also conducted by means of avidin and biotin binding.

As a method of screening for proteins, for example, that bind to the MLX polypeptide using any of the MLX polypeptides described above, many methods well known by a person skilled in the art can be used. Such a screening can be conducted by, for example, immunoprecipitation method, specifically, in the following manner.

In immunoprecipitation, an immune complex is formed by adding an antibody to cell lysate prepared using an appropriate detergent. The antibody used in the immunoprecipitation for the screening recognizes any of the MLX proteins 1-163. Alternatively, when an MLX protein fused with a recognition site (epitope) is used in the screening, antibodies against the epitope may be used for the immunoprecipitaion. The immune complex consists of the MLX protein, a polypeptide comprising the binding ability with the MLX protein, and an antibody.

An immune complex can be precipitated, for example by Protein A sepharose or Protein G sepharose when the antibody is a mouse IgG antibody. If the MLX polypeptide is prepared as a fusion protein with an epitope, such as GST, an immune complex can be formed in the same manner as in the use of the antibody against the MLX polypeptide, using a substance specifically binding to these epitopes, such as glutathione-Sepharose 4B.

Immunoprecipitation can be performed by following or according to, for example, the methods in the literature (Harlow and Lane, Antibodies, 511-52, Cold Spring Harbor Laboratory publications, New York (1988)).

SDS-PAGE is commonly used for analysis of immunoprecipitated proteins and the bound protein can be analyzed by the molecular weight of the protein using gels with an appropriate concentration. Since the protein bound to the MLX polypeptide is difficult to detect by a common staining method, such as Coomassie staining or silver staining, the detection sensitivity for the protein can be improved by culturing cells in culture medium containing radioactive isotope, ³⁵S-methionine or ³⁵S-cystein, labeling proteins in the cells, and detecting the proteins. The target protein can be purified directly from the SDS-polyacrylamide gel and its sequence can be determined, when the molecular weight of a protein has been revealed.

As a method for screening proteins binding to the MLX polypeptide using the polypeptide, for example, West-Western blotting analysis (Skolnik et al., Cell 65: 83-90 (1991)) can be used. Specifically, a protein binding to the MLX polypeptide can be obtained by preparing a cDNA library from cells, tissues, organs or cultured cells expected to express a protein binding to the MLX polypeptide using a phage vector (e.g., ZAP), expressing the protein on LB-agarose, fixing the protein expressed on a filter, reacting the purified and labeled MLX polypeptide with the above filter, and detecting the plaques expressing proteins bound to the MLX polypeptide according to the label. The MLX polypeptide may be labeled by utilizing the binding between biotin and avidin, or by utilizing an antibody that specifically binds to the MLX polypeptide, or a peptide or polypeptide (for example, GST) that is fused to the MLX polypeptide. Methods using labeling substances such as radioisotope (e.g., ³H, ¹⁴C, ³²P, ³³P, ³⁵S, ¹²⁵I, ¹³¹I), enzymes (e.g., alkaline phosphatase, horseradish peroxidase, β-galactosidase, β-glucosidase), fluorescent substances (e.g., fluorescein isothiosyanete (FITC), rhodamine) and biotin/avidin, may be used for the labeling in the present method. When the MLX protein is labeled with radioisotope, the detection or measurement can be carried out by liquid scintillation. Alternatively, MLX proteins labeled with enzymes can be detected or measured by adding a substrate of the enzyme to detect the enzymatic change of the substrate, such as generation of color, with absorptiometer. Further, in case where a fluorescent substance is used as the label, the bound protein may be detected or measured using fluorophotometer.

Alternatively, in another embodiment of the screening method of the present invention, a two-hybrid system utilizing cells may be used (“MATCHMAKER Two-Hybrid system”, “Mammalian MATCHMAKER Two-Hybrid Assay Kit”, “MATCHMAKER one-Hybrid system” (Clontech); “HybriZAP Two-Hybrid Vector System” (Stratagene); the references “Dalton and Treisman, Cell 68: 597-612 (1992)”, “Fields and Sternglanz, Trends Genet 10: 286-92 (1994)”).

In the two-hybrid system, the MLX polypeptide is fused to the SRF-binding region or GALA-binding region and expressed in yeast cells. A cDNA library is prepared from cells expected to express a protein binding to the MLX polypeptide, such that the library, when expressed, is fused to the VP16 or GAL4 transcriptional activation region. The cDNA library is then introduced into the above yeast cells and the cDNA derived from the library is isolated from the positive clones detected (when a protein binding to the MLX polypeptide is expressed in yeast cells, the binding of the two activates a reporter gene, making positive clones detectable). A protein encoded by the cDNA can be prepared by introducing the cDNA isolated above to E. coli and expressing the protein.

As a reporter gene, for example, Ade2 gene, lacZ gene, CAT gene, luciferase gene and such can be used besides HIS3 gene.

A compound binding to the MLX polypeptide can also be screened using affinity chromatography. For example, the MLX polypeptide may be immobilized on a carrier of an affinity column, and a test compound, containing a protein capable of binding to the MLX polypeptide, is applied to the column. A test compound herein may be, for example, cell extracts, cell lysates, etc. After loading the test compound, the column is washed, and compounds bound to the MLX polypeptide can be prepared.

When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon may be used as a mean for detecting or quantifying the bound compound in the present invention. When such a biosensor is used, the interaction between the MLX polypeptide and a test compound can be observed real-time as a surface plasmon resonance signal, using only a minute amount of polypeptide and without labeling (for example, BIAcore, Pharmacia). Therefore, it is possible to evaluate the binding between the MLX polypeptide and a test compound using a biosensor such as BIAcore.

The methods of screening for molecules that bind when the immobilized MLX polypeptide is exposed to synthetic chemical compounds, or natural substance banks, or a random phage peptide display library, or the methods of screening using high-throughput based on combinatorial chemistry techniques (Wrighton et al., Science 273: 458-64 (1996); Verdine, Nature 384: 11-13 (1996); Hogan, Nature 384: 17-9 (1996)) to isolate not only proteins but chemical compounds that bind to the MLX protein (including agonist and antagonist) are well known to those skilled in the art.

A compound isolated by the screening is a candidate for drugs which promote or inhibit the activity of the MLX polypeptide, for treating colorectal cancer or preventing metastasis of colorectal cancer. A compound in which a part of the structure of the compound obtained by the present screening method having the activity of binding to the MLX polypeptide is converted by addition, deletion and/or replacement, is included in the compounds obtained by the screening method of the present invention.

Alternatively, when the biological activity of the MLX polypeptide is detected in the screening of the present invention, a compound isolated by this screening is a candidate for agonists or antagonists of the MLX polypeptide. The term “agonist” refers to molecules that activate the function of the MLX polypeptide by binding thereto. Likewise, the term “antagonist” refers to molecules that inhibit the function of the MLX polypeptide by binding thereto. Moreover, a compound isolated by this screening is a candidate for compounds which inhibit the in vivo interaction of the MLX polypeptide with molecules (including DNAs and proteins).

When the biological activity to be detected in the present method is cell proliferation, it can be detected, for example, by preparing cells which express the MLX polypeptide, culturing the cells in the presence of a test compound, and determining the speed of cell proliferation, measuring the cell cycle and such, as well as by measuring the colony forming activity.

A compound isolated by the above screenings is a candidate for drugs which inhibit the activity of the MLX polypeptide and can be applied for the treatment of colorectal cancer and the prevention of metastasis of colorectal cancer. Moreover, compound in which a part of the structure of the compound inhibiting the activity of the MLX protein is converted by addition, deletion and/or replacement are also included in the compounds obtainable by the screening method of the present invention.

In a further embodiment, the present invention provides methods for screening candidate agents which are potential targets in the treatment of colorectal cancer and prevention of metastasis of colorectal cancer. The method is based on screening a candidate therapeutic agent to determine if it converts an expression profile of MLX 1-163 sequences characteristic of primary colorectal cancer with metastatic lesions to a pattern indicative of a colorectal cancer state without metastatic lesions. As discussed in detail above, by controlling the expression levels of the MLX 1-163, one can control the onset and progression of colorectal cancer and metastasis of colorectal cancer. Thus, candidate agents, which are potential targets in the treatment of colorectal cancer or prevention of metastasis of colorectal cancer, can be identified through screenings that use the expression levels and activities of the MLX polypeptide as indices. In the context of the present invention, such screening may comprise, for example, the following steps: (a) contacting a test compound with a cell expressing one or more marker genes; and (b) selecting a compound that reduces the expression level of the marker gene in comparison with the expression level detected in the absence of the test compound.

Cells expressing at least one of the marker genes include, for example, cell lines established from colorectal cancer, preferably from primary colorectal cancer cell. For example, the cell is an immortalized cell line derived from a primary colorectal cancer cell. The marker genes for the screening are selected from the group of genes encoding MLXs 1-163.

The expression level can be estimated by methods well known to those skilled in the art. In the method of screening, a compound that reduces the expression level of at least one of the MLX genes can be selected as candidate agents. A decrease in expression compared to the normal control level indicates the agent is an inhibitor of metastatic lesions of colorectal cancer associated up-regulated gene and useful to inhibit development of metastatic lesions of colorectal cancer. An agent effective in suppressing expression of overexpressed genes is deemed to lead to a clinical benefit, and such compounds may be further tested for the ability to inhibit metastasis or cancer cell growth.

Furthermore, based on this screening method, using a test cell population from a subject as the cell expressing one or more marker genes, therapeutic agents for treating colorectal cancer or preventing metastasis of colorectal cancer that is appropriate for the subject, i.e., a particular individual can be selected.

Differences in the genetic makeup of individuals can result in differences in their relative abilities to metabolize various drugs. An agent that is metabolized in a subject to act as an anti-colorectal cancer agent can manifest itself by inducing a change in gene expression pattern in the subject's cells from the characteristic of a metastatic state to a gene expression pattern characteristic of a non-metastatic state. Accordingly, the differentially expressed MLX sequences disclosed herein allow for a putative therapeutic or prophylactic anti-colorectal cancer agent to be tested in a test cell population from a selected subject in order to determine if the agent is a suitable anti-colorectal cancer agent in the subject.

To identify an anti-colorectal cancer agent, that is appropriate for a specific subject, a test cell population from the subject is exposed to a test compound, and the expression of one or more of MLX 1-163 sequences is determined.

The test cell population contains primary lesions of colorectal cancer cells expressing metastasis-associated gene. Preferably, the test cell is an epithelial cell. For example a test cell population is incubated in the presence of a test compound and the pattern of gene expression of one or more of MLX 1-163 sequences in the test cell population is measured and compared to one or more reference profiles, e.g., reference expression profile of primary colorectal cancer with metastasis or non-metastatic colorectal cancer reference expression profile. An increase in expression of one or more of the sequences MLX 1-163 in a test cell population relative to a reference cell population containing metastatic lesions of colorectal cancer is indicative that the agent is therapeutic.

Further, in another embodiment of the method of screening for a compound for treating colorectal cancer or preventing metastasis of colorectal cancer, the method utilizes the promoter region of an MLX gene. Compounds inhibiting the expression of the MLX gene in colorectal cancer cells are expected to serve as candidates for drugs that can be applied to the treatment of diseases associated with the MLX polypeptide, for example, colorectal carcinoma, and to the prevention of metastasis of colorectal carcinoma.

This screening method includes the steps of: (1) constructing a vector comprising the transcriptional regulatory region of a gene selected from the group consisting of MLXs 1-163 upstream of a reporter gene; (2) transforming a cell with the vector of step (1); (3) contacting a test compound with the cell of step (2); (4) detecting the expression of the reporter gene; and (5) selecting the test compound that suppresses the expression of the reporter gene compared to that in the absence of the test compound.

The transcriptional regulatory region of an MLX gene can be obtained from genomic libraries using the 5′ region of the human MLX genes (MLX 1-163; see Table 1) as the probe. Any reporter gene may be used in the screening so long as its expression can be detected in the screening. Example of reporter genes include the β-gal gene, the CAT gene, and the luciferase gene. Detection of the expression of the reporter gene can be conducted corresponding to the type of the reporter gene. Although there are no particular restriction on the cell into which the vector is introduced, preferable examples include cells derived from primary lesions of colorectal cancer with metastasis.

The compound isolated by the screening is a candidate for drugs which inhibit the expression of an MLX protein and can be applied to the treatment of colorectal cancer or prevention of metastasis of colorectal cancer. Moreover, compounds in which a part of the structure of the compound inhibiting the transcriptional activation of the MLX protein is converted by addition, deletion, substitution and/or insertion are also included in the compounds obtainable by the screening method of the present invention.

Any test compound, for example, cell extracts, cell culture supernatant, products of fermenting microorganism, extracts from marine organism, plant extracts, purified or crude proteins, peptides, non-peptide compounds, synthetic micromolecular compounds and natural compounds, can be used in the screening methods of the present invention. The test compound of the present invention can be also obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des 12: 145 (1997)). Examples of methods for the synthesis of molecular libraries can be found in the art, for example in: DeWitt et al., Proc Natl Acad Sci USA 90: 6909 (1993); Erb et al., Proc Natl Acad Sci USA 91: 11422 (1994); Zuckermann et al., J Med Chem 37: 2678 (1994); Cho et al., Science 261: 1303 (1993); Carrell et al., Angew Chem Int Ed Engl 33: 2059 (1994); Carell et al., Angew Chem Int Ed Engl 33: 2061 (1994); Gallop et al., J Med Chem 37:1233 (1994). Libraries of compounds may be presented in solution (e.g., Houghten, Bio Techniques 13: 412 (1992)), or on beads (Lam, Nature 354: 82 (1991)), chips (Fodor, Nature 364: 555 (1993)), bacteria (U.S. Pat. No. 5,223,409), spores (U.S. Pat. Nos. 5,571,698; 5,403,484; and 5,223,409), plasmids (Cull et al., Proc Natl Acad Sci USA 89: 1865 (1992)) or phage (Scott and Smith, Science 249: 386 (1990); Devlin, Science 249: 404 (1990); Cwirla et al., Proc Natl Acad Sci USA 87: 6378 (1990); Felici, J Mol Biol 222: 301 (1991); United States Patent Application 20020103360).

Kits

The invention also includes an MLX-detection reagent, e.g., a nucleic acid that specifically binds to or identifies one or more MLX nucleic acids such as oligonucleotide sequences, which are complementary to a portion of an MLX nucleic acid. The reagents are packaged together in the form of a kit. The reagents are packaged in separate containers, e.g., a nucleic acid (either bound to a solid matrix or packaged separately with reagents for binding them to the matrix), a control reagent (positive and/or negative), and/or a detectable label. Instructions (e.g., written, tape, VCR, CD-ROM, etc.) for carrying out the assay may be included in the kit. The assay format of the kit is, for example, Northern hybridization.

For example, MLX detection reagent is immobilized on a solid matrix such as a porous strip to form at least one MLX detection site. The measurement or detection region of the porous strip may include a plurality of sites containing a nucleic acid. A test strip may also contain sites for negative and/or positive controls. Alternatively, control sites are located on a separate strip from the test strip. Optionally, the different detection sites may contain different amounts of immobilized nucleic acids, i.e., a higher amount in the first detection site and lesser amounts in subsequent sites. Upon the addition of test sample, the number of sites displaying a detectable signal provides a quantitative indication of the amount of MLX present in the sample. The detection sites may be configured in any suitably detectable shape and are typically in the shape of a bar or dot spanning the width of a teststrip.

Alternatively, the kit contains a nucleic acid substrate array comprising one or more nucleic acid sequences. The nucleic acids on the array specifically identify one or more nucleic acid sequences represented by MLXs 1-163. The expression of 2, 3, 4, 5, 6, 7, 8, 9, 10, 15, 20, 25, 40 or 50 or more of the sequences represented by MLX 1-163 are identified by virtue if the level of binding to an array test strip or chip. The substrate array can be on, e.g., a solid substrate, e.g., a “chip” as described in U.S. Pat. No. 5,744,305.

Array and Pluralities

The invention also includes a nucleic acid substrate array comprising one or more nucleic acid sequences. The nucleic acids on the array specifically corresponds to one or more nucleic acid sequences represented by MLX 1-163. The expression level of 2, 3, 4, 5, 6, 7, 8, 9, 10, 15, 20, 25, 40 or 50 or more of the sequences represented by MLX 1-163 are identified by detecting nucleic acid binding to the array.

The invention also includes an isolated plurality (i.e., a mixture of two or more nucleic acids) of nucleic acid sequences. The nucleic acid sequences are in a liquid phase or a solid phase, e.g., immobilized on a solid support such as a nitrocellulose membrane. The plurality includes one or more of the nucleic acid sequences represented by MLX 1-163. In various embodiments, the plurality includes 2, 3, 4, 5, 6, 7, 8, 9, 10, 15, 20, 25, 40 or 50 or more of the sequences represented by MLX 1-163.

Chips

The DNA chip is a device that is convenient to compare expression levels of a number of genes at the same time. DNA chip-based expression profiling can be carried out, for example, by the method as disclosed in “Microarray Biochip Technology” (Mark Schena, Eaton Publishing, 2000), etc.

A DNA chip comprises immobilized high-density probes to detect a number of genes. Thus, expression levels of many genes can be estimated at the same time by a single-round analysis. Namely, the expression profile of a specimen can be determined with a DNA chip. The DNA chip-based method of the present invention comprises the following steps of:
(1) synthesizing aRNAs or cDNAs corresponding to the marker genes;
(2) hybridizing the aRNAs or cDNAs with probes for marker genes; and
(3) detecting the aRNA or cDNA hybridizing with the probes and quantifying the amount of mRNA thereof.

The aRNA refers to RNA transcribed from a template cDNA with RNA polymerase. A aRNA transcription kit for DNA chip-based expression profiling is commercially available. With such a kit, aRNA can be synthesized from T7 promoter-attached cDNA as a template using T7 RNA polymerase. On the other hand, by PCR using random primer, cDNA can be amplified using as a template a cDNA synthesized from mRNA.

On the other hand, the DNA chip comprises probes, which have been spotted thereon, to detect the marker genes of the present invention. There is no limitation on the number of marker genes spotted on the DNA chip. For example, it is allowed to select 5% or more, preferably 20% or more, more preferably 50% or more, still more preferably 70% or more of the marker genes of the present invention. Any other genes as well as the marker genes can be spotted on the DNA chip. For example, a probe for a gene whose expression level is hardly altered may be spotted on the DNA chip. Such a gene can be used to normalize assay results when assay results are intended to be compared between multiple chips or between different assays.

A probe is designed for each marker gene selected, and spotted on a DNA chip. Such a probe may be, for example, an oligonucleotide comprising 5-50 nucleotide residues. A method for synthesizing such oligonucleotides on a DNA chip is known to those skilled in the art. Longer DNAs can be synthesized by PCR or chemically. A method for spotting long DNA, which is synthesized by PCR or the like, onto a glass slide is also known to those skilled in the art. A DNA chip that is obtained by the method as described above can be used for diagnosing metastasis of colorectal cancer according to the present invention.

The prepared DNA chip is contacted with aRNA, followed by the detection of hybridization between the probe and aRNA. The aRNA can be previously labeled with a fluorescent dye. A fluorescent dye such as Cy3 (red) and Cy5 (green) can be used to label a aRNA. aRNAs from a subject and a control are labeled with different fluorescent dyes, respectively. The difference in the expression level between the two can be estimated based on a difference in the signal intensity. The signal of fluorescent dye on the DNA chip can be detected by a scanner and analyzed using a special program. For example, the Suite from Affymetrix is a software package for DNA chip analysis.

Methods for Treating Colorectal Cancer or Preventing Metastasis of Colorectal Cancer

The invention provides a method for alleviating a symptom of colorectal cancer, inhibiting tumor growth or proliferation of primary lesions of colorectal cancer, or inhibiting metastasis of colorectal cancer in a subject. Therapeutic compounds are administered prophylactically or therapeutically to subject suffering from or at risk of (or susceptible to) developing metastatic lesions of colorectal cancer. Such subjects are identified using standard clinical methods or by detecting an aberrant level of expression or activity of a metastasis-associated gene, e.g., MLX 1-163. Prophylactic administration occurs prior to the manifestation of overt clinical symptoms of disease, such that a disease or disorder is prevented or, alternatively, delayed in its progression.

The phrase “inhibiting metastasis of colorectal cancer” herein includes inhibition of development of metastatic lesions; wherein development of metastatic lesions encompass generation of metastatic lesions and promotion of metastasis, such as release of cancer cells from the primary site, intravasation to neighboring vessels, transport to the site of metastasis through blood flow, and extravasation and/or infarction to the distant organ as described above.

The method includes decreasing the expression, or function, or both, of one or more gene products of genes whose expression is aberrantly increased (“overexpressed gene”). The expression is inhibited in any of several ways known in the art. For example, the expression is inhibited by administering to the subject a compound screened by the screening method of the present invention.

Alternatively, the expression may be inhibited by administering to the subject a nucleic acid that inhibits or antagonizes, the expression of the overexpressed gene or genes, e.g., an antisense oligonucleotide or small interference RNA (siRNA) which disrupts expression of the overexpressed gene or genes.

Such nucleic acids include polynucleotides which specifically hybridize with the polynucleotide encoding human MLX or the complementary strand thereof, and which comprises at least 15 nucleotides. The phrase “specifically hybridize” as used herein, means that cross-hybridization does not occur significantly with DNA encoding other proteins, under the usual hybridizing conditions, preferably under stringent hybridizing conditions.

Preferable nucleic acids that inhibit one or more gene products of overexpressed genes include an antisense oligonucleotide that hybridizes with any site within the nucleotide sequence encoding an MLX protein. This antisense oligonucleotide is preferably against at least 15 continuous nucleotides of the nucleotide sequence encoding an MLX protein. The above-mentioned antisense oligonucleotide, which contains an initiation codon in the above-mentioned at least 15 continuous nucleotides, is even more preferred.

Derivatives or modified products of antisense oligonucleotides can be used as antisense oligonucleotides. Examples of such modified products include lower alkyl phosphonate modifications such as methyl-phosphonate-type or ethyl-phosphonate-type, phosphorothioate modifications and phosphoroamidate modifications.

The term “antisense oligonucleotides” as used herein means, not only those in which the nucleotides corresponding to those constituting a specified region of a DNA or mRNA are entirely complementary, but also those having a mismatch of one or more nucleotides, as long as the DNA or mRNA and the antisense oligonucleotide can specifically hybridize with the nucleotide sequence encoding an MLX protein.

Polynucleotides are contained as those having, in the “at least 15 continuous nucleotide sequence region,” when they have a homology of at least 70% or higher, preferably at 80% or higher, more preferably 90% or higher, even more preferably 95% or higher. The algorithm stated herein can be used to determine the homology.

The antisense oligonucleotide derivatives act upon cells producing the MLX polypeptide by binding to the DNA or mRNA encoding the MLX polypeptide, inhibiting its transcription or translation, promoting the degradation of the mRNA, and inhibiting the expression of the MLX polypeptide, thereby resulting in the inhibition of the MLX polypeptide's function.

The nucleic acids that inhibit one or more gene products of overexpressed genes also include small interfering RNAs (siRNA) comprising a combination of a sense strand nucleic acid and an antisense strand nucleic acid of the nucleotide sequence encoding an MLX protein.

The term “siRNA” refers to a double stranded RNA molecule which prevents translation of a target mRNA. Standard techniques are used for introducing siRNA into cells, including those wherein DNA is used as the template to transcribe RNA. The siRNA comprises a sense nucleic acid sequence and an anti-sense nucleic acid sequence of the polynucleotide encoding a human MLX protein. The siRNA is constructed such that a single transcript (double stranded RNA) has both the sense and complementary antisense sequences from the target gene, e.g., a hairpin.

The method is used to suppress gene expression of a cell with up-regulated expression of an MLX gene. Binding of the siRNA to the MLX gene transcript in the target cell results in a reduction of MLX protein production by the cell. The length of the oligonucleotide is at least 10 nucleotides and may be as long as the naturally occurring transcript. Preferably, the oligonucleotide is 19-25 nucleotides in length. Most preferably, the oligonucleotide is less than 75, 50 or 25 nucleotides in length.

The nucleotide sequence of siRNAs may be designed using an siRNA design computer program available from the Ambion website (http://www.ambion.com/techlib/misc/siRNA_finder.html). Nucleotide sequences for the siRNA are selected by the computer program based on the following protocol:

Selection of siRNA Target Sites:
1. Beginning with the AUG start codon of transcript, scan downstream for AA dinucleotide sequences. Record the occurrence of each AA and the 3′ adjacent 19 nucleotides as potential siRNA target sites. Tuschl, et al. recommend not to design siRNA against the 5′ and 3′ untranslated regions (UTRs) and regions near the start codon (within 75 bases) as these may be richer in regulatory protein binding sites, and thus the complex of endonuclease and siRNAs that were designed against these regions may interfere with the binding of UTR-binding proteins and/or translation initiation complexes.
2. Compare the potential target sites to the human genome database and eliminate from consideration any target sequences with significant homology to other coding sequences. The homology search can be performed using BLAST, which can be found on the NCBI server at: www.ncbi.nlm.nih.gov/BLAST/
3. Select qualifying target sequences for synthesis. On the website of Ambion, several preferable target sequences can be selected along the length of the gene for evaluation.

Alternatively, function of one or more gene products of the overexpressed genes is inhibited by administering a compound that binds to or otherwise inhibits the function of the gene products. For example, the compound is an antibody which binds to the overexpressed gene product or gene products.

An antibody that binds to the MLX polypeptide may be in any form, such as monoclonal or polyclonal antibodies, and includes antiserum obtained by immunizing an animal such as a rabbit with the MLX polypeptide, all classes of polyclonal and monoclonal antibodies, human antibodies and humanized antibodies produced by genetic recombination.

An MLX polypeptide used as an antigen to obtain an antibody may be derived from any animal species, but preferably is derived from a mammal such as a human, mouse or rat, more preferably from a human. A human-derived polypeptide may be obtained from the nucleotide or amino acid sequences disclosed herein (see, Table 1).

According to the present invention, the polypeptide to be used as an immunization antigen may be a complete protein or a partial peptide of the protein. A partial peptide may comprise, for example, the amino (N)-terminal or carboxy (C)-terminal fragment of an MLX polypeptide. Herein, an antibody is defined as a protein that reacts with either the full length or a fragment of an MLX polypeptide.

A gene encoding an MLX polypeptide or its fragment may be inserted into a known expression vector, which is then used to transform a host cell as described herein. The desired polypeptide or its fragment may be recovered from the outside or inside of host cells by any standard method, and may subsequently be used as an antigen. Alternatively, whole cells expressing the polypeptide or their lysates, or a chemically synthesized polypeptide may be used as the antigen.

Any mammalian animal may be immunized with the antigen, but preferably the compatibility with parental cells used for cell fusion is taken into account. In general, animals of Rodentia, Lagomorpha or Primates are used. Animals of Rodentia include, for example, mouse, rat and hamster. Animals of Lagomorpha include, for example, rabbit. Animals of Primates include, for example, a monkey of Catarrhini (old world monkey) such as Macaca fasciculans, rhesus monkey, sacred baboon and chimpanzees.

Methods for immunizing animals with antigens are known in the art. Intraperitoneal injection or subcutaneous injection of antigens is a standard method for immunization of mammals. More specifically, antigens may be diluted and suspended in an appropriate amount of phosphate buffered saline (PBS), physiological saline, etc. If desired, the antigen suspension may be mixed with an appropriate amount of a standard adjuvant, such as Freund's complete adjuvant, made into emulsion, and then administered to mammalian animals. Preferably, it is followed by several administrations of antigen mixed with an appropriately amount of Freund's incomplete adjuvant every 4 to 21 days. An appropriate carrier may also be used for immunization. After immunization as above, serum is examined by a standard method for an increase in the amount of desired antibodies.

Polyclonal antibodies against the MLX polypeptides may be prepared by collecting blood from the immunized mammal examined for the increase of desired antibodies in the serum, and by separating serum from the blood by any conventional method. Polyclonal antibodies include serum containing the polyclonal antibodies, as well as the fraction containing the polyclonal antibodies may be isolated from the serum. Immunoglobulin G or M can be prepared from a fraction which recognizes only the MLX polypeptide using, for example, an affinity column coupled with the MLX polypeptide, and further purifying this fraction using protein A or protein G column.

To prepare monoclonal antibodies, immune cells are collected from the mammal immunized with the antigen and checked for the increased level of desired antibodies in the serum as described above, and are subjected to cell fusion. The immune cells used for cell fusion are preferably obtained from spleen. Other preferred parental cells to be fused with the above immunocyte include, for example, myeloma cells of mammalians, and more preferably myeloma cells having an acquired property for the selection of fused cells by drugs.

The above immunocyte and myeloma cells can be fused according to known methods, for example, the method of Milstein et al. (Galfre and Milstein, Methods Enzymol 73: 346 (1981)).

Resulting hybridomas obtained by the cell fusion may be selected by cultivating them in a standard selection medium, such as HAT medium (hypoxanthine, aminopterin and thymidine containing medium). The cell culture is typically continued in the HAT medium for several days to several weeks, the time being sufficient to allow all the other cells, with the exception of the desired hybridoma (non-fused cells), to die. Then, the standard limiting dilution is performed to screen and clone a hybridoma cell producing the desired antibody.

In addition to the above method, in which a non-human animal is immunized with an antigen for preparing hybridoma, human lymphocytes such as those infected by EB virus may be immunized with a polypeptide, polypeptide expressing cells, or their lysates in vitro. Then, the immunized lymphocytes are fused with human-derived myeloma cells that are capable of indefinitely dividing, such as U266, to yield a hybridoma producing a desired human antibody that is able to bind to the MLX polypeptide can be obtained (Unexamined Published Japanese Patent Application No. (JP-A) Sho 63-17688).

The obtained hybridomas are subsequently transplanted into the abdominal cavity of a mouse and the ascites are extracted. The obtained monoclonal antibodies can be purified by, for example, ammonium sulfate precipitation, a protein A or protein G column, DEAE ion exchange chromatography, or an affinity column to which the MLX polypeptide is coupled. The antibody serve as a candidate for agonists and antagonists of the MLX polypeptide and can be applied to the antibody treatment for diseases related to the MLX polypeptide. When the obtained antibody is to be administered to the human body (antibody treatment), a human antibody or a humanized antibody is preferable for reducing immunogenicity.

For example, transgenic animals having a repertory of human antibody genes may be immunized with an antigen selected from a polypeptide, polypeptide expressing cells or their lysates. Antibody producing cells are then collected from the animals and fused with myeloma cells to obtain hybridoma, from which human antibodies against the polypeptide can be prepared (see WO92-03918, WO93-2227, WO94-02602, WO94-25585, WO96-33735 and WO96-34096).

Alternatively, an immune cell such as an immunized lymphocyte, producing antibodies may be immortalized by an oncogene and used for preparing monoclonal antibodies.

Monoclonal antibodies thus obtained can be also recombinantly prepared using genetic engineering techniques (see, for example, Borrebaeck and Larnick, Therapeutic Monoclonal Antibodies, published in the United Kingdom by MacMillan Publishers LTD (1990)). For example, a DNA encoding an antibody may be cloned from an immune cell, such as a hybridoma or an immunized lymphocyte producing the antibody, inserted into an appropriate vector, and introduced into host cells to prepare a recombinant antibody.

Furthermore, an antibody used for the method of treating colorectal cancer or preventing metastasis of colorectal cancer of the present invention may be a fragment of an antibody or modified antibody, so long as it binds to one or more of the MLX polypeptides. For instance, the antibody fragment may be Fab, F(ab′)₂, Fv or single chain Fv (scFv), in which Fv fragments from H and L chains are ligated by an appropriate linker (Huston et al, Proc Natl Acad Sci USA 85:5879-83 (1988)). More specifically, an antibody fragment may be generated by treating an antibody with an enzyme, such as papain or pepsin. Alternatively, a gene encoding the antibody fragment may be constructed, inserted into an expression vector, and expressed in an appropriate host cell (see, for example, Co et al., J Immunol 152: 2968-76 (1994); Better and Horwitz, Methods Enzymol 178: 476-96 (1989); Pluckthun and Skerra, Methods Enzymol 178: 497-515 (1989); Lamoyi, Methods Enzymol 121: 652-63 (1986); Rousseaux et al., Methods Enzymol 121: 663-9 (1986); Bird and Walker, Trends Biotechnol 9: 132-7 (1991)).

An antibody may be modified by conjugation with a variety of molecules, such as polyethylene glycol (PEG). The modified antibody can be obtained by chemically modifying an antibody. These modification methods are conventional in the field.

Alternatively, an antibody may be obtained as a chimeric antibody, between a variable region derived from nonhuman antibody and the constant region derived from human antibody, or as a humanized antibody, comprising the complementarity determining region (CDR) derived from nonhuman antibody, the frame work region (FR) derived from human antibody, and the constant region. Such antibodies can be prepared using known technology.

Antibodies obtained as above may be purified to homogeneity. For example, the separation and purification of the antibody can be performed according to separation and purification methods used for general proteins. For example, the antibody may be separated and isolated by the appropriately selected and combined use of column chromatographies, such as affinity chromatography, filter, ultrafiltration, salting-out, dialysis, SDS polyacrylamide gel electrophoresis, isoelectric focusing and others (Antibodies: A Laboratory Manual. Ed Harlow and David Lane, Cold Spring Harbor Laboratory (1988)), but are not limited thereto. A protein A column and protein G column can be used as the affinity column. Exemplary protein A columns to be used include, for example, Hyper D, POROS and Sepharose F.F. (Pharmacia).

Exemplary chromatography, with the exception of affinity includes, for example, ion-exchange chromatography, hydrophobic chromatography, gel filtration, reverse-phase chromatography, adsorption chromatography and the like (Strategies for Protein Purification and Characterization: A Laboratory Course Manual. Ed Daniel R. Marshak et al., Cold Spring Harbor Laboratory Press (1996)). The chromatographic procedures can be carried out by liquid-phase chromatography, such as HPLC and FPLC.

For example, measurement of absorbance, enzyme-linked immunosorbent assay (ELISA), enzyme immunoassay (EIA), radioimmunoassay (RIA), and/or immunofluorescence may be used to measure the antigen binding activity of the antibody against an MLX protein. In ELISA, the antibody is immobilized on a plate, an MLX polypeptide is applied to the plate, and then a sample containing a desired antibody, such as culture supernatant of antibody producing cells or purified antibodies, is applied. Then, a secondary antibody that recognizes the primary antibody and is labeled with an enzyme, such as alkaline phosphatase, is applied, and the plate is incubated. Next, after washing, an enzyme substrate, such as p-nitrophenyl phosphate, is added to the plate, and the absorbance is measured to evaluate the antigen binding activity of the sample. A fragment of the polypeptide, such as a C-terminal or N-terminal fragment, may be used as the antigen to evaluate the binding activity of the antibody. BIAcore (Pharmacia) may be used to evaluate the activity of the antibody against an MLX protein.

The present invention provides a method for treating colorectal cancer or preventing metastasis of colorectal cancer, using an antibody against an MLX polypeptide. According to the method, a pharmaceutically effective amount of an antibody against the MLX polypeptide is administered. Since the expression of the MLX protein is up-regulated in colorectal cancer cells with metastasis, and the suppression of the expression of these proteins is expected to lead to the decrease in metastasis, or cell proliferating or growing activity, it is expected that colorectal cancer can be treated or prevented, or metastasis of colorectal cancer can be suppressed or prevented by binding the antibody and these proteins. Thus, an antibody against an MLX polypeptide are administered at a dosage sufficient to reduce the activity of the MLX protein. Alternatively, an antibody binding to a cell surface marker specific for tumor cells can be used as a tool for drug delivery. Thus, for example, an antibody against an MLX polypeptide conjugated with a cytotoxic agent may be administered at a dosage sufficient to injure tumor cells.

Furthermore, the present invention provides a method for treating colorectal cancer or preventing metastasis of colorectal cancer by administering an MLX polypeptide or a polynucleotide encoding the polypeptide. The MLX proteins and immunologically active fragments thereof are useful as vaccines against colorectal cancer or metastasis of colorectal cancer. Thus, the present invention also relates to a method of inducing anti-tumor immunity comprising the step of administering an MLX protein or an immunologically active fragment thereof, a polynucleotide encoding the protein or fragments thereof, or a vector comprising the polynucleotide or fragments thereof. In some cases the proteins or fragments thereof may be administered in a form bound to the T cell recepor (TCR) or presented by an antigen presenting cell (APC), such as macrophage, dendritic cell (DC) or B-cells. Due to the strong antigen presenting ability of DC, the use of DC is most preferable among the APCs.

In the present invention, vaccine against colorectal cancer or metastasis of colorectal cancer refers to a substance that has the function to induce anti-tumor immunity or immunity to suppress metastasis upon inoculation into animals. In general, anti-tumor immunity includes immune responses such as follows:
induction of cytotoxic lymphocytes against tumors,
induction of antibodies that recognize tumors, and
induction of anti-tumor cytokine production.

Therefore, when a certain protein induces any one of these immune responses upon inoculation into an animal, the protein is decided to have anti-tumor immunity inducing effect. The induction of the anti-tumor immunity by a protein can be detected by observing in vivo or in vitro the response of the immune system in the host against the protein.

For example, a method for detecting the induction of cytotoxic T lymphocytes is well known. A foreign substance that enters the living body is presented to T cells and B cells by the action of antigen presenting cells (APCs). T cells that respond to the antigen presented by APC in antigen specific manner differentiate into cytotoxic T cells (or cytotoxic T lymphocytes; CTLs) due to stimulation by the antigen, and then proliferate (this is referred to as activation of T cells). Therefore, CTL induction by a certain peptide can be evaluated by presenting the peptide to T cell by APC, and detecting the induction of CTL. Furthermore, APC has the effect of activating CD4+ T cells, CD8+ T cells, macrophages, eosinophils, and NK cells. Since CD4+ T cells and CD8+ T cells are also important in anti-tumor immunity, the anti-tumor immunity inducing action of the peptide can be evaluated using the activation effect of these cells as indicators.

A method for evaluating the inducing action of CTL using dendritic cells (DCs) as APC is well known in the art. DC is a representative APC having the strongest CTL inducing action among APCs. In this method, the test polypeptide is initially contacted with DC, and then this DC is contacted with T cells. Detection of T cells having cytotoxic effects against the cells of interest after the contact with DC shows that the test polypeptide has an activity of inducing the cytotoxic T cells. Activity of CTL against tumors can be detected, for example, using the lysis of ⁵¹Cr-labeled tumor cells as the indicator. Alternatively, the method of evaluating the degree of tumor cell damage using ³H-thymidine uptake activity or LDH (lactose dehydrogenase)-release as the indicator is also well known.

Apart from DC, peripheral blood mononuclear cells (PBMCs) may also be used as the APC. The induction of CTL is reported that it can be enhanced by culturing PBMC in the presence of GM-CSF and IL4. Similarly, CTL has been shown to be induced by culturing PBMC in the presence of keyhole limpet hemocyanin (KLH) and IL-7.

The test polypeptides confirmed to possess CTL inducing activity by these methods are polypeptides having DC activation effect and subsequent CTL inducing activity. Therefore, polypeptides that induce CTL against tumor cells are useful as vaccines against tumors. Furthermore, APC that acquired the ability to induce CTL against tumors by contacting with the polypeptides are useful as vaccines against tumors. Furthermore, CTL that acquired cytotoxicity due to presentation of the polypeptide antigens by APC can be also used as vaccines against tumors. Such therapeutic methods for tumors using anti-tumor immunity due to APC and CTL are referred to as cellular immunotherapy.

Generally, when using a polypeptide for cellular immunotherapy, efficiency of the CTL-induction is known to increase by combining a plurality of polypeptides having different structures and contacting them with DC. Therefore, when stimulating DC with protein fragments, it is advantageous to use a mixture of multiple types of fragments.

Alternatively, the induction of anti-tumor immunity by a polypeptide can be confirmed by observing the induction of antibody production against tumors. For example, when antibodies against a polypeptide are induced in a laboratory animal immunized with the polypeptide, and when growth, proliferation or metastasis of tumor cells is suppressed by those antibodies, the polypeptide can be determined to have an ability to induce anti-tumor immunity.

Anti-tumor immunity is induced by administering the vaccine of this invention, and the induction of anti-tumor immunity enables treatment of colorectal cancer and prevention of metastasis of colorectal cancer. Therapy against cancer, or prevention of the onset of cancer or metastasis of cancer includes any of the steps, such as inhibition of the growth of cancerous cells, involution of cancer, suppression of occurrence of cancer, and metastasis of cancer. Decrease in mortality of individuals having cancer, decrease of tumor markers in the blood, alleviation of detectable symptoms accompanying cancer, and such are also included in the therapy or prevention of cancer. Such therapeutic and preventive effects are preferably statistically significant. For example, in observation, at a significance level of 5% or less, wherein the therapeutic or preventive effect of a vaccine against cell proliferative diseases is compared to a control without vaccine administration. For example, Student's t-test, the Mann-Whitney U-test or ANOVA may be used for statistical analyses.

The above-mentioned protein having immunological activity, or a polynucleotide or vector encoding the protein may be combined with an adjuvant. An adjuvant refers to a compound that enhances the immune response against the protein when administered together (or successively) with the protein having immunological activity. Examples of adjuvants include cholera toxin, salmonella toxin, alum and such, but are not limited thereto. Furthermore, the vaccine of this invention may be combined appropriately with a pharmaceutically acceptable carrier. Examples of such carriers are sterilized water, physiological saline, phosphate buffer, culture fluid and such. Furthermore, the vaccine may contain as necessary, stabilizers, suspensions, preservatives, surfactants and such. The vaccine is administered systemically or locally. Vaccine administration may be performed by single administration or boosted by multiple administrations.

When using APC or CTL as the vaccine of this invention, tumors can be treated or prevented, for example, by the ex vivo method. More specifically, PBMCs of the subject receiving treatment or prevention are collected, the cells are contacted with the polypeptide ex vivo, and following the induction of APC or CTL, the cells may be administered to the subject. APC can be also induced by introducing a vector encoding the polypeptide into PBMCs ex vivo. APC or CTL induced in vitro can be cloned prior to administration. By cloning and growing cells having high activity of damaging target cells, cellular immunotherapy can be performed more effectively. Furthermore, APC and CTL isolated in this manner may be used for cellular immunotherapy not only against individuals from whom the cells are derived, but also against similar types of tumors from other individuals.

Composition for Treating Colorectal Cancer or Preventing Metastasis of Colorectal Cancer

When administrating the compound isolated by the screening methods of the invention as a pharmaceutical for humans and other mammals, such as mice, rats, guinea-pigs, rabbits, chicken, cats, dogs, sheep, pigs, cattle, monkeys, baboons or chimpanzees, for treating colorectal cancer or preventing metastasis of colorectal cancer the isolated compound can be directly administered or can be formulated into a dosage form using known pharmaceutical preparation methods.

Pharmaceutical formulations include those suitable for oral, rectal, nasal, topical (including buccal and sub-lingual), vaginal or parenteral (including intramuscular, sub-cutaneous and intravenous) administration, or for administration by inhalation or insufflation. The formulations are optionally packaged in discrete dosage units.

Pharmaceutical formulations suitable for oral administration include capsules, cachets or tablets, each containing a predetermined amount of the active ingredient. Formulations also include powders, granules or solutions, suspensions or emulsions. The active ingredient is optionally administered as a bolus electuary or paste. Tablets and capsules for oral administration may contain conventional excipients such as binding agents, fillers, lubricants, disintegrant or wetting agents. A tablet may be made by compression or molding, optionally with one or more formulational ingredients. Compressed tablets may be prepared by compressing in a suitable machine the active ingredients in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may be coated according to methods well known in the art. Oral fluid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils) or preservatives. The tablets may optionally be formulated so as to provide slow or controlled release of the active ingredient therein. A package of tablets may contain one tablet to be taken on each of the month.

Formulations for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit dose or multi-dose containers, for example sealed ampoules and vials, and may be stored in a freeze-dried (lyophilized) condition requiring only the addition of the sterile liquid carrier, for example, saline, water-for-injection, immediately prior to use. Alternatively, the formulations may be presented for continuous infusion. Extemporaneous injection solutions and suspensions may be prepared from sterile powders, granules and tablets of the kind previously described.

Formulations for rectal administration include suppositories with standard carriers such as cocoa butter or polyethylene glycol. Formulations for topical administration in the mouth, for example buccally or sublingually, include lozenges, which contain the active ingredient in a flavored base such as sucrose and acacia or tragacanth, and pastilles comprising the active ingredient in a base such as gelatin and glycerin or sucrose and acacia. For intra-nasal administration the compounds of the invention may be used as a liquid spray or dispersible powder or in the form of drops. Drops may be formulated with an aqueous or non-aqueous base also comprising one or more dispersing agents, solubilizing agents or suspending agents.

For administration by inhalation the compounds are conveniently delivered from an insufflator, nebulizer, pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflators.

Other formulations include implantable devices and adhesive patches; which release a therapeutic agent.

When desired, the above-described formulations, adapted to give sustained release of the active ingredient, may be employed. The pharmaceutical compositions may also contain other active ingredients such as antimicrobial agents, immunosuppressants or preservatives.

It should be understood that in addition to the ingredients particularly mentioned above, the formulations of this invention may include other agents conventional in the art having regard to the type of formulation in question, for example, those suitable for oral administration may include flavoring agents, surfactants, stabilizers, excipients, vehicles, preservatives, binders and such, in a unit dose form required for generally accepted drug implementation.

Methods well known to one skilled in the art may be used to administer the inventive pharmaceutical compound to patients, for example as intraarterial, intravenous, percutaneous injections and also as intranasal, transbronchial, intramuscular or oral administrations. The dosage and method of administration vary according to the body-weight and age of a patient and the administration method; however, one skilled in the art can routinely select them. If said compound is encodable by a DNA, the DNA can be inserted into a vector for gene therapy and the vector administered to perform the therapy. The dosage and method of administration vary according to the body-weight, age, and symptoms of a patient but one skilled in the art can select them suitably.

For example, although there are some differences according to the symptoms, the dose of a compound that binds with the polypeptide of the present invention and regulates its activity is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kgs of body-weight.

The present invention provides a composition for treating colorectal cancer or preventing metastasis of colorectal cancer using an antisense oligonucleotide derivative or siRNA derivative against one or more MLX genes as the active ingredients. The derivatives can be made into an external preparation, such as a liniment or a poultice, by mixing with a suitable base material which is inactive against the derivatives.

Also, as needed, the derivatives can be formulated into tablets, powders, granules, capsules, liposome capsules, injections, solutions, nose-drops and freeze-drying agents by adding excipients, isotonic agents, solubilizers, stabilizers, preservatives, pain-killers and such. These can be prepared by following usual methods.

The antisense oligonucleotide derivative or siRNA derivative is given to the patient by directly applying onto the ailing site or by injecting into a blood vessel so that it will reach the site of ailment. A mounting medium can also be used to increase durability and membrane-permeability. Examples are, liposome, poly-L-lysine, lipid, cholesterol, lipofectin or derivatives of these.

The dosage of the antisense oligonucleotide derivative or siRNA derivative of the present invention can be adjusted suitably according to the patient's condition and used in desired amounts. For example, a dose range of 0.1 to 100 mg/kg, preferably 0.1 to 50 mg/kg can be administered.

The present invention further provides a composition for treating colorectal cancer or preventing metastasis of colorectal cancer by administering an antibody against an MLX protein or fragment thereof to a subject.

Furthermore, a composition for treating colorectal cancer or preventing metastasis of colorectal cancer, comprising a pharmaceutically effective amount of an MLX polypeptide is provided. The composition comprising the MLX protein may be used for raising anti tumor immunity. Moreover, in place of an MLX protein, polynucleotides or vectors encoding the MLX protein may be administered to the subject for treating colorectal cancer and preventing metastasis of colorectal cancer. The form of the polynucleotides and vectors encoding the MLX protein is not restricted in any way so long as they express the MLX protein or fragments thereof in the subject and induce anti-tumor immunity in the subject.

For example, although there are some differences according to the symptoms, the dose of an antibody or polypeptide for treating colorectal cancer or preventing metastasis of colorectal cancer is about 0.1 mg to about 100 mg per day, preferably about 1.0 mg to about 50 mg per day and more preferably about 1.0 mg to about 20 mg per day, when administered orally to a normal adult (weight 60 kg).

When administering parenterally, in the form of an injection to a normal adult (weight 60 kg), although there are some differences according to the patient, target organ, symptoms and method of administration, it is convenient to intravenously inject a dose of about 0.01 mg to about 30 mg per day, preferably about 0.1 to about 20 mg per day and more preferably about 0.1 to about 10 mg per day. Also, in the case of other animals too, it is possible to administer an amount converted to 60 kgs of body-weight.

The following examples are presented to illustrate the present invention and to assist one of ordinary skill in making and using the same. The examples are not intended in any way to otherwise limit the scope of the invention.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. Any patents, patent applications and publications cited herein are incorporated by reference.

### BEST MODE FOR CARRYING OUT THE INVENTION

The present invention is illustrated in details by following Examples, but is not restricted to these Examples.

1. Materials and Methods

(1) Tissue Samples and Laser-Capture Microdissection (LCM)

Primary CRC tissues and, their corresponding non-cancerous mucosae were obtained with informed consent from 15 patients who underwent colectomy and hepatectomy in the same operation. Primary CRC tissues without liver metastasis and their corresponding non-cancerous mucosae were obtained with informed consent from eleven patients who underwent colectomy. Nine benign adenomas were obtained from either the resected specimen of the cancer patients or tumors from endoscopic polypectomy with informed consent. All of the samples were imbedded in TissueTek OCT medium (Sakura, Tokyo, Japan) and frozen at −80° C. Later, the frozen sections were fixed in 70% ethanol for 45 sec., stained with hematoxylin and eosin, and dehydrated in 70:30, 50:50, and 30:70 of ethanol: xylene for 30 sec. in each step, followed by a final dehydration in 100% xylene for two min. Upon air-drying, the stained tissues were microdissected using PixCell LCM system (Arcturus Engineering, Mountain View, Calif.) according to the manufacturer's protocols. Cancerous cells from the primary lesions were selectively microdissected (˜2×10⁴ cells from each sample).

(2) RNA Extraction and T7-Based RNA Amplification

Total RNAs were extracted from each sample of the laser-captured cells into 350 μl of RLT lysis buffer (QIAGEN, Hilden, Germany). The extracted RNAs were treated for 1 h at 37° C. with 10 units of DNase I (Roche, Basel, Switzerland) in the presence of 1U of RNase inhibitor (TOYOBO, Osaka, Japan) to remove any contaminating genomic DNAs. After inactivation at 70° C. for 10 min, the RNAs were purified with RNeasy Mini Kit (QIAGEN) according to the manufacturer's recommendations. All DNase I-treated RNAs were subjected to T7-based amplification as described previously (Ono et al., Cancer Res 60: 5007-11 (2000)). Two rounds of amplification yielded 15-80 μg of amplified RNA (aRNA) from each sample.

(3) Construction and Analysis of cDNA Microarray

23040 independent cDNAs were selected, including some ESTs, from the UniGene database of the National Center for Biotechnology Information. The DNA spotted on the microarray slides were prepared by RT-PCR using sets of gene-specific primers and a mixture of commercially provided poly A RNAs (Clontech, Palo Alto, Calif.) as a template (Ono et al., Cancer Res 60: 5007-11 (2000)). The products were applied to electrophoresis on agarose gels and those showing a single band of expected size were utilized for spotting. Further sequence analyses of randomly selected 2485 products from 23040 genes collaborated the complete concordance of their cDNA sequences.

Duplicate sets of cDNA spots were used for each analysis of expression profiles, to reduce experimental fluctuation. Three-microgram aliquots of aRNA from each primary tumor and normal epithelium were labeled respectively with Cy3-dCTP and Cy5-dCTP (Amersham Pharmacia Biotech) to compare the expression between primary lesion and non-cancerous mucosa. Equal amounts of Cy3- and Cy5-labeled probes were co-hybridized onto the microarray slides. Hybridization, washing, and scanning were performed as described previously (Ono et al., Cancer Res 60: 5007-11 (2000)).

(4) Data Analysis

The intensity of each duplicated signal was evaluated photometrically by the Array Vision computer program (Imaging Research Inc., St. Catharines, Ontario, Canada) and normalized so that the averaged Cy3/Cy5-ratio of 52 housekeeping genes that had been spotted on the microarray slides was 1.0 (Kitahara et al., Cancer Res 61: 3544-9 (2001); Ono et al., Cancer Res 60: 5007-11 (2000)). Because data derived from low signal intensities are less reliable, cut-off values for signal intensities were determined on each slide so that all filtered genes have greater S/N (signal to noise) ratios of Cy3 or Cy5 than three and excluded genes for further analysis when both Cy3 and Cy5 dyes gave signal intensities lower than the cut-off. The Cy3/Cy5 ratio for each gene was calculated by averaging duplicate spots (Kitahara et al., Cancer Res 61: 3544-9 (2001); Ono et al., Cancer Res 60: 5007-11 (2000)). In respect to the comparison between non-cancerous mucosae and primary tumors, genes were categorized into three groups according to their expression ratios (Cy3/Cy5): up-regulated (ratio equal to or greater than 2.0), down-regulated (ratio equal to or less than 0.5), and unchanged expression (ratios between 0.5 and 2.0). Genes with Cy3/Cy5 ratios greater than 2.0 or less than 0.5 in more than 50% of the cases examined were defined as frequently up- or down-regulated genes, respectively.

2. Results

(1) Isolation of Primary CRCs by LCM

To obtain precise expression profiles of primary and metastatic cancer cells, laser-capture microdissection (LCM) was employed to collect pure populations of each type. The proportion of cancer cells selected by this procedure was estimated to be >95%, as determined by microscopic visualization (data not shown).

(2) Identification of Genes Frequently Up-Regulated in Primary CRCs with Liver Metastasis but not in those without Metastasis or Premalignant Tumors

In addition, expression profiles of the 15 primary colon cancers were compared with their corresponding non-cancerous mucosae. This analysis identified a large number of genes that are frequently elevated in the cancer tissues. First, expression profiles of 11 advanced colorectal carcinomas without liver metastasis, 9 premalignant adenomas and the corresponding non-cancerous mucosae were analyzed. To identify novel diagnostic markers for the prediction of metastatic potential of the tumor cells from the primary lesion, next genes whose expression levels were different between primary lesions with metastasis and those without metastasis were selected. The expression profiles of the 15 CRCs with liver metastasis were compared to those of the 20 lesions without metastasis. Genes for which up-regulated expression could be observed in more than half of the primary lesions with liver metastasis and in less than 20% of tumors without metastasis were selected. The criteria identified 163 genes including 10 ESTs (Table 1). Since approximately 30% of patients with advanced colorectal cancer have disease recurrence in their liver after the removal of primary tumors, expression of these genes may serve for predictive markers of liver metastasis

TABLE 1
Frequently Up-regulated Genes in CRCs with Liver Metastasis

| MLX | | | | | |
|---|---|---|---|---|---|
| Assignment | LMMID | Symbol | Title | ACCESSION | Unigene-ID |
| 1 | A7124 | DRIL1 | dead ringer (Drosophila)-like 1 | U88047 | 198515 |
| 2 | A9574 | LOC51082 | RNA polymerase I 16 kDa | AA865274 | 106127 |
| | | | subunit | | |
| 3 | A6321 | TIM17B | translocase of inner | AJ005895 | 19105 |
| | | | mitochondrial membrane 17 | | |
| | | | (yeast) homolog B | | |
| 4 | A0148 | LYN | v-yes Yamaguchi sarcoma | M16038 | 80887 |
| | | | viral related oncogene | | |
| | | | homolog | | |
| 5 | A7546 | RIPK2 | receptor-interacting | AC004003 | 103755 |
| | | | serine-threonine kinase 2 | | |
| 6 | A4602 | TRAM | translocating | X63679 | 4147 |
| | | | chain-associating membrane | | |
| | | | protein | | |
| 7 | B2224 | | EST | AA683373 | 117208 |
| 8 | A1131 | HRIHFB2206 | HRIHFB2206 protein | L10378 | 82508 |
| | | | glyceronephosphate | | |
| 9 | A2228 | GNPAT | O-acyltransferase | AJ002190 | 12482 |
| 10 | E0641 | COP9 | COP9 homolog | AW162338 | 75193 |
| 11 | A1371 | | KIAA0111 gene product | D21853 | 79768 |
| 12 | A4420 | CACYBP | calcyclin binding protein | AF057356 | 27258 |
| 13 | A8813 | MRPL30 | mitochondrial ribosomal | AA668892 | 12094 |
| | | | protein 30 | | |
| 14 | A0490 | MIF | macrophage migration | L10612 | 73798 |
| | | | inhibitory factor | | |
| | | | (glycosylation-inhibiting | | |
| | | | factor) | | |
| 15 | A1619 | CETN2 | centrin, EF-hand protein, 2zd79f02.r1 | X72964 | 82794 |
| | | | Homo sapiens | | |
| 16 | C4909 | | cDNA clone IMAGE: 346875 | W79821 | 284207 |
| | | | 5′ | | |
| 17 | B4837 | LBR | lamin B receptor | L25941 | 152931 |
| 18 | A4472 | SH3BGRL | SH3 domain binding glutamic | AF042081 | 14368 |
| | | | acid-rich protein like | | |
| 19 | A8172 | | KIAA0877 protein | N79367 | 11217 |
| 20 | A7204 | TXN | thioredoxin | AA315827 | 76136 |
| 21 | C8557 | TMEPAI | transmembrane, prostate | AA536113 | 83883 |
| | | | androgen induced RNA | | |
| 22 | A3152 | ASCL1 | achaete-scute complex | L08424 | 1619 |
| | | | (Drosophila) homolog-like 1 | | |
| 23 | A2571 | AVPR1A | arginine vasopressin receptor | U19906 | 2131 |
| | | | 1A | | |
| 24 | A6101 | | Homo sapiens cDNA: | N35843 | 184793 |
| | | | FLJ21880 fis, clone | | |
| | | | HEP02743 | | |
| 25 | A3947 | SEP15 | 15 kDa selenoprotein | AF051894 | 90606 |
| 26 | A5355 | JCL | hepatocellular carcinoma | AA478499 | 4943 |
| | | | associated protein; breast | | |
| | | | cancer associated gene 1 | | |
| 27 | A3970 | GPR49 | G protein-coupled receptor 49 | AF062006 | 246996 |
| 28 | B5140 | NME4 | non-metastatic cells 4, protein | AI360872 | 9235 |
| | | | expressed in | | |
| 29 | A5663 | HSPC125 | HSPC125 protein | N79130 | 5232 |
| 30 | B4964 | BZW2 | basic leucine zipper and W2 | D60918 | 5216 |
| | | | domains 2 | | |
| 31 | A0289 | MCM6 | minichromosome | U46838 | 155462 |
| | | | maintenance deficient (mis5, | | |
| | | | S. pombe) 6 | | |
| 32 | B8316 | HSPC023 | HSPC023 protein | AI268685 | 279945 |
| 33 | A5676 | MRPL47 | mitochondrial ribosomal | W74502 | 283734 |
| | | | protein L47 | | |
| 34 | A9074 | DREV1 | CGI-81 protein | W19984 | 279583 |
| 35 | A0290 | MTX1 | metaxin 1 | U46920 | 247551 |
| 36 | A4351 | PIK3R2 | phosphoinositide-3-kinase, | X80907 | 211586 |
| | | | regulatory subunit, | | |
| | | | polypeptide 2 (p85 beta) | | |
| 37 | B3939 | CTNNAL1 | catenin (cadherin-associated | U97067 | 58488 |
| | | | protein), alpha-like 1 | | |
| 38 | A1181 | TIEG | TGFB inducible early growth | U21847 | 82173 |
| | | | response | | |
| 39 | A2898 | CBX3 | chromobox homolog 3 | U26312 | 8123 |
| | | | (Drosophila HP1 gamma) | | |
| 40 | A3555 | TK1 | thymidine kinase 1, soluble | K02581 | 105097 |
| 41 | C0745 | | ESTs, Moderately similar to | W72297 | 31965 |
| | | | RS20_HUMAN 40S | | |
| | | | RIBOSOMAL PROTEIN S2 | | |
| | | | [H. sapiens] | | |
| 42 | A7677 | MSH6 | mutS (E. coli) homolog 6 | U28946 | 3248 |
| 43 | A0240N | PTK9 | protein tyrosine kinase 9 | N86645 | 82643 |
| 44 | A5741 | | KIAA0776 protein | D20853 | 5460 |
| 45 | D5349 | ASNS | asparagine synthetase | AI025236 | 75692 |
| 46 | A2250 | SFRS1 | splicing factor, | M72709 | 73737 |
| | | | arginine/serine-rich 1 | | |
| | | | (splicing factor 2, alternate | | |
| | | | splicing factor) | | |
| 47 | A2076 | PRCP | prolylcarboxypeptidase | L13977 | 75693 |
| | | | (angiotensinase C) | | |
| 48 | A6711 | | hypothetical protein | T70782 | 8173 |
| | | | FLJ10803 | | |
| 49 | D7803 | SLC11A2 | solute carrier family 11 | N20907 | 57435 |
| | | | (proton-coupled divalent | | |
| | | | metal ion transporters), | | |
| | | | member 2 | | |
| 50 | B4668 | | hypothetical protein | AI341719 | 26006 |
| | | | FLJ10559 | | |
| 51 | A9431 | RPL26L1 | ribosomal protein L26 | AI023779 | 110165 |
| | | | homolog | | |
| 52 | A3928 | SDCCAG8 | serologically defined colon | AF039690 | 171409 |
| | | | cancer antigen 8 | | |
| 53 | A7218 | HCCS | holocytochrome c synthase | U36787 | 211571 |
| | | | (cytochrome c heme-lyase) | | |
| 54 | A2944 | NDUFS5 | NADH dehydrogenase | AF047434 | 80595 |
| | | | (ubiquinone) Fe—S protein 5 | | |
| | | | (15 kD) (NADH-coenzyme Q | | |
| | | | reductase) | | |
| 55 | A2000 | MEST | mesoderm specific transcript | D78611 | 79284 |
| | | | (mouse) homolog | | |
| 56 | A2875 | DAD1 | defender against cell death 1 | D15057 | 82890 |
| 57 | A0521 | NFATC3 | nuclear factor of activated | U14510 | 172674 |
| | | | T-cells, cytoplasmic, | | |
| | | | calcineurin-dependent 3 | | |
| 58 | A2109 | RPS5 | ribosomal protein S5 | U14970 | 76194 |
| 59 | A2170 | CCT3 | chaperonin containing TCP1, | X74801 | 1708 |
| | | | subunit 3 (gamma) | | |
| 60 | B1933 | | EST | AA663135 | 116888 |
| 61 | A3260 | NHP2L1 | non-histone chromosome | D50420 | 182255 |
| | | | protein 2 (S. cerevisiae)-like 1 | | |
| 62 | A2171 | CCT3 | chaperonin containing TCP1, | X74801 | 1708 |
| | | | subunit 3 (gamma) | | |
| 63 | A4307 | PGRMC1 | progesterone receptor | Y12711 | 90061 |
| | | | membrane component 1 | | |
| 64 | A3311 | RPS21 | ribosomal protein S21 | L04483 | 1948 |
| 65 | A0507N | PC4 | activated RNA polymerase II | AI308856 | 74861 |
| | | | transcription cofactor 4 | | |
| 66 | D1304 | RNPC2 | RNA-binding region | AA460324 | 145696 |
| | | | containing 2 | | |
| 67 | A6752 | RPL14 | ribosomal protein L14 | U16738 | 158675 |
| 68 | B2830 | | ESTs | AA127750 | 118650 |
| 69 | B4681 | | hypothetical protein | AA088410 | 26369 |
| | | | FLJ20287 | | |
| 70 | A3800 | SPHAR | S-phase response | X82554 | 296169 |
| | | | (cyclin-related) | | |
| 71 | A2173 | KCNA3 | potassium voltage-gated | M85217 | 169948 |
| | | | channel, shaker-related | | |
| | | | subfamily, member 3 | | |
| 72 | C7864 | SLC35A2 | solute carrier family 35 | D84454 | 21899 |
| | | | (UDP-galactose transporter), | | |
| | | | member 2 | | |
| 73 | A4608 | CTSC | cathepsin C | X87212 | 10029 |
| 74 | A1544 | NPY5R | neuropeptide Y receptor Y5 | U56079 | 158330 |
| 75 | A5153 | ZNF282 | zinc finger protein 282 | D30612 | 58167 |
| 76 | A3353 | FBRNP | heterogeneous nuclear protein | S63912 | 249247 |
| | | | similar to rat helix | | |
| | | | destabilizing protein | | |
| 77 | A5665 | FABP1 | fatty acid binding protein 1, | AA001405 | 5241 |
| | | | liver | | |
| 78 | A2374 | TAF2F | TATA box binding protein | X97999 | 155188 |
| | | | (TBP)-associated factor, RNA | | |
| 79 | A1325 | PSORT | polymerase II, F, 55 kD | AF050198 | 129730 |
| | | | putative mitochondrial space | | |
| | | | protein 32.1 | | |
| 80 | A2527 | PSME1 | proteasome (prosome, | L07633 | 75348 |
| | | | macropain) activator subunit 1 | | |
| | | | (PA28 alpha) | | |
| 81 | A5324 | CDKN2C | cyclin-dependent kinase | AI357641 | 4854 |
| | | | inhibitor 2C (p18, inhibits | | |
| | | | CDK4) | | |
| 82 | A2479 | GPD1 | glycerol-3-phosphate | L34041 | 25478 |
| | | | dehydrogenase 1 (soluble) | | |
| 83 | A3245 | UBE1 | ubiquitin-activating enzyme | M58028 | 2055 |
| | | | E1 (A1S9T and BN75 | | |
| | | | temperature sensitivity | | |
| | | | complementing) | | |
| 84 | A2231 | AP2M1 | adaptor-related protein | D63475 | 152936 |
| | | | complex 2, mu 1 subunit | | |
| 85 | B0918 | LOC51637 | CGI-99 protein | AA576803 | 110803 |
| 86 | A0467 | RPL19 | ribosomal protein L19 | X63527 | 75879 |
| 87 | A3153 | EFNA1 | ephrin-A1 | M57730 | 1624 |
| 88 | B1087 | P17.3 | neuronal protein 17.3 | AI360274 | 111497 |
| 89 | B4538 | PCNP | PEST-containing nuclear | AA232823 | 283728 |
| | | | protein | | |
| 90 | B8804 | G2AN | alpha glucosidase II alpha | D42041 | 76847 |
| | | | subunit | | |
| 91 | B4136 | TAF2F | TATA box binding protein | U18062 | 155188 |
| | | | (TBP)-associated factor, RNA | | |
| | | | polymerase II, F, 55 kD | | |
| 92 | A0851 | PDHB | pyruvate dehydrogenase | M34479 | 979 |
| | | | (lipoamide) beta | | |
| 93 | B2937 | H2AFZ | H2A histone family, member Z | AA416820 | 119192 |
| 94 | C1070 | | ESTs | W51962 | 34447 |
| 95 | A5764 | HMG2 | high-mobility group | AC002400 | 80684 |
| | | | (nonhistone chromosomal) | | |
| | | | protein 2 | | |
| 96 | C4139 | | ESTs | AI039201 | 54548 |
| 97 | B4362 | | Homo sapiens cDNA | AA453271 | 301728 |
| | | | FLJ20642 fis, clone | | |
| | | | KAT02751 | | |
| 98 | A7435N | IGFBP2 | insulin-like growth factor | X16302 | 162 |
| | | | binding protein 2 (36 kD) | | |
| 99 | A9274 | CDC42 | cell division cycle 42 | N63172 | 146409 |
| | | | (GTP-binding protein, 25 kD) | | |
| 100 | A2498 | SHMT2 | serine | L11932 | 75069 |
| | | | hydroxymethyltransferase 2 | | |
| | | | (mitochondrial) | | |
| 101 | A6703 | SAS10 | disrupter of silencing 10 | AI346431 | 87627 |
| 102 | B6456 | FBXW1B | f-box and WD-40 domain | AI092703 | 21229 |
| | | | protein 1B | | |
| 103 | E1234 | HMG1L10 | high-mobility group | AI927968 | 274472 |
| | | | (nonhistone chromosomal) | | |
| | | | protein 1-like 10 | | |
| 104 | B3750 | SEC63L | SEC63, endoplasmic | AA233728 | 31575 |
| | | | reticulum translocon | | |
| | | | component (S. cerevisiae) like | | |
| | | | homolog of mouse quaking | | |
| 105 | B2475 | QKI | QKI (KH domain RNA | AA768310 | 15020 |
| | | | binding protein) | | |
| 106 | A2024 | GCL | grancalcin | M81637 | 79381 |
| 107 | E0653 | | hypothetical protein | BG166302 | 8886 |
| | | | FLJ20424 | | |
| 108 | A9040 | SLC2A1 | solute carrier family 2 | K03195 | 169902 |
| | | | (facilitated glucose | | |
| | | | transporter), member 1 | | |
| 109 | B4066 | CLIC1 | chloride intracellular channel 1 | AA291390 | 74276 |
| 110 | A7453N | DOC1 | deleted in oral cancer (mouse, | AA315033 | 3436 |
| | | | homolog) 1 | | |
| 111 | A2411N | MRPS24 | mitochondrial ribosomal | AI312652 | 284286 |
| | | | protein S24 | | |
| 112 | A0268 | COPS5 | COP9 (constitutive | U70734 | 198767 |
| | | | photomorphogenic, | | |
| | | | Arabidopsis, homolog) | | |
| | | | subunit 5 | | |
| 113 | B5278 | | hypothetical protein | AA098911 | 9911 |
| | | | FLJ11773 | | |
| 114 | A4826 | HRMT1L2 | HMT1 (hnRNP | D66904 | 20521 |
| | | | methyltransferase, S. cerevisiae)- | | |
| | | | like 2 | | |
| 115 | C5006 | PREP | prolyl endopeptidase | X74496 | 86978 |
| 116 | C2106 | | ESTs | AA936889 | 301786 |
| 117 | A3375 | EIF1A | eukaryotic translation | L18960 | 4310 |
| | | | initiation factor 1A | | |
| | | | Homo sapiens cDNA: | | |
| | | | FLJ22662 fis, clone | | |
| 118 | A5870 | | HSI08080 | W76303 | 178470 |
| 119 | A8558 | TMPO | thymopoietin | AA811648 | 11355 |
| 120 | D1507 | LOC54543 | 6.2 kd protein | AA961415 | 112318 |
| 121 | A3615 | TIEG | TGFB inducible early growth | U21847 | 82173 |
| | | | response | | |
| 122 | A0740N | BMI1 | murine leukemia viral (bmi) | L13689 | 431 |
| | | | oncogene homolog | | |
| 123 | D6450 | TMP21 | transmembrane trafficking | AI261341 | 74137 |
| | | | protein | | |
| 124 | A1156N | AMFR | autocrine motility factor | M63175 | 80731 |
| | | | receptor | | |
| 125 | A5119 | GCAT | glycine C-acetyltransferase | AF077740 | 54609 |
| | | | (2-amino-3-ketobutyrate | | |
| | | | coenzyme A ligase) | | |
| 126 | B2482 | | ESTs, Highly similar to | AI125528 | 166253 |
| | | | KIAA0476 protein | | |
| | | | [H. sapiens] | | |
| 127 | B2668 | | ESTs | N63894 | 118142 |
| 128 | B5455 | NUBP2 | nucleotide binding protein 2 | AA847227 | 256549 |
| | | | (E. coli MinD like) | | |
| 129 | A2850 | SNRPA1 | small nuclear | X13482 | 80506 |
| | | | ribonucleoprotein polypeptide | | |
| | | | A′ | | |
| 130 | A2400 | XRCC5 | X-ray repair complementing | M30938 | 84981 |
| | | | defective repair in Chinese | | |
| | | | hamster cells 5 | | |
| | | | (double-strand-break | | |
| | | | rejoining; Ku autoantigen, | | |
| | | | 80 kD) | | |
| 131 | A0447 | RPS29 | ribosomal protein S29 | U14973 | 539 |
| 132 | A4142 | IDH2 | isocitrate dehydrogenase 2 | AA573936 | 5337 |
| | | | (NADP+), mitochondrial | | |
| 133 | A5181 | OMI | HtrA-like serine protease | AF020760 | 115721 |
| 134 | A0372 | STIP1 | stress-induced-phosphoprotein | M86752 | 75612 |
| | | | 1 (Hsp70/Hsp90-organizing | | |
| | | | protein) | | |
| 135 | A2394 | HNRPC | heterogeneous nuclear | M16342 | 182447 |
| | | | ribonucleoprotein C (C1/C2) | | |
| 136 | B4116 | RPL19 | ribosomal protein L19 | X63527 | 75879 |
| 137 | B4141 | | KIAA0172 protein | AA885708 | 77546 |
| 138 | A0192 | EGR1 | early growth response 1 | M62829 | 738 |
| 139 | C4968 | | DKFZP564B167 protein | AI192351 | 76285 |
| 140 | A1848 | ANXA4 | annexin A4 | M19383 | 77840 |
| | | | Human DNA sequence from | | |
| | | | PAC 130G2 on chromosome | | |
| | | | 6p22.2-22.3. Contains | | |
| | | | ribosomal protein L29 | | |
| 141 | B7739 | | pseudogene, ESTs and STSs | AL008627 | 166181 |
| 142 | A8879N | HCA112 | hepatocellular | AA583491 | 12126 |
| | | | carcinoma-associated antigen | | |
| | | | 112 | | |
| 143 | A4388 | EVPL | envoplakin | U53786 | 25482 |
| 144 | A5339 | SARS | seryl-tRNA synthetase | AA703770 | 4888 |
| 145 | A1703 | STX5A | syntaxin 5A | U26648 | 154546 |
| 146 | C4988 | SNW1 | SKI-INTERACTING | W20455 | 79008 |
| | | | PROTEIN | | |
| 147 | A4272 | T54 | T54 protein | AI201465 | 100391 |
| | | | hypothetical protein | | |
| 148 | B0460 | | FLJ11088 | D20934 | 227591 |
| 149 | A2151 | LGALS8 | lectin, galactoside-binding, | L78132 | 4082 |
| | | | soluble, 8 (galectin 8) | | |
| 150 | A1176 | MYO1C | myosin IC | U14391 | 82251 |
| | | | hypothetical protein | | |
| 151 | B3852N | | FLJ10661 | AA416981 | 279033 |
| 152 | A1041 | PTS | 6-pyruvoyltetrahydropterin | U63383 | 366 |
| | | | synthase | | |
| 153 | B4110 | GTF3C2 | general transcription factor | D13636 | 75782 |
| | | | IIIC, polypeptide 2 (beta | | |
| | | | subunit, 110 kD) | | |
| 154 | E0757 | SSA2 | Sjogren syndrome antigen A2 | AW079181 | 554 |
| | | | (60 kD, ribonucleoprotein | | |
| | | | autoantigen SS-A/Ro) | | |
| 155 | A9468 | | ESTs | AA452295 | 110406 |
| | | | Homo sapiens cDNA: | | |
| | | | FLJ21869 fis, clone | | |
| 156 | B9606 | | HEP02442 | AA884861 | 28465 |
| | | | hypothetical protein | | |
| 157 | E0571 | | FLJ10726 | AW243438 | 268561 |
| 158 | B4483 | NLVCF | nuclear localization signal | AI052044 | 19500 |
| | | | deleted in velocardiofacial | | |
| | | | syndrome | | |
| 159 | A0429 | UBCH10 | ubiquitin carrier protein E2-C | U73379 | 93002 |
| 160 | A5411 | C15orf12 | chromosome 15 open reading | W76431 | 6118 |
| | | | frame 12 | | |
| 161 | A9830 | VCP | valosin-containing protein | AA614311 | 106357 |
| 162 | B3800 | CABC1 | chaperone, ABC1 activity of | AA477232 | 273186 |
| | | | bc1 complex like | | |
| | | | thyroid hormone receptor | | |
| 163 | A1808N | AIB3 | binding protein | W92633 | 159613 |

### INDUSTRIAL APPLICABILITY

The expression of MLX nucleic acids of the present invention was frequently elevated in the primary lesions compared to their corresponding non-cancerous mucosae. Accordingly, these genes may serve as a diagnostic marker of metastasis of colorectal cancers.

While the invention has been described in detail and with reference to specific embodiments thereof, it will be apparent to one skilled in the art that various changes and modifications can be made therein without departing from the spirit and scope of the invention.

1. A method of diagnosing a predisposition to developing metastatic lesions of colorectal cancer in a subject, comprising determining a level of expression of metastasis-associated gene in a patient derived biological sample, wherein an increase in said level compared to a normal control level of said gene indicates that said subject suffers from or is at risk of developing metastatic lesions of colorectal cancer.

2. The method of claim 1, wherein said metastasis-associated gene is selected from the group consisting of MLXs 1-163.

3. The method of claim 1, wherein said method further comprises determining said level of expression of a plurality of metastasis-associated genes.

4. The method of claim 1, wherein the expression level of metastasis-associated gene is determined by any one method selected from the group consisting of:
(a) detecting mRNA of the metastasis-associated gene;
(b) detecting protein encoded by the metastasis-associated gene; and
(c) detecting the biological activity of the protein encoded by the metastasis-associated gene.

5. The method of claim 1, wherein said level of expression is determined by detecting hybridization of metastasis-associated gene probe to a gene transcript of said patient derived biological sample.

6. The method of claim 5, wherein said hybridization step is carried out on a DNA chip.

7. The method of claim 1, wherein said patient derived biological sample is primary colorectal cancer.

8. The method of claim 1, wherein said increase is at least 10% greater than said normal control level.

9. A primary colorectal cancer reference expression profile, comprising a pattern of gene expression of two or more genes selected from the group consisting of MLXs 1-163.

10. A method of screening for a compound for treating colorectal cancer or preventing metastasis of colorectal cancer, said method comprising the steps of:
(1) contacting a test compound with a polypeptide selected from the group consisting of:
(a) a polypeptide comprising the amino acid sequence encoded by a polynucleotide selected from the group consisting of MLXs 1-163;
(b) a polypeptide that comprises the amino acid sequence encoded by a polynucleotide selected from the group consisting of MLXs 1-163, in which one or more amino acids are substituted, deleted, inserted, and/or added and that has a biological activity equivalent to a protein consisting of the amino acid sequence encoded by the polynucleotide; and
(c) a polypeptide encoded by a polynucleotide that hybridizes under stringent conditions to a polynucleotide selected from the group consisting of MLXs 1-163, wherein the polypeptide has a biological activity equivalent to a polypeptide consisting of the amino acid sequence encoded by the polynucleotide selected from the group consisting of MLXs 1-163;
(2) detecting the binding activity between the polypeptide and the test compound; and
(3) selecting a compound that binds to the polypeptide.

11. A method of screening for a compound for treating colorectal cancer or preventing metastasis of colorectal cancer, said method comprising the steps of:
(1) contacting a test compound with a polypeptide selected from the group consisting of:
(a) a polypeptide comprising the amino acid sequence encoded by a polynucleotide selected from the group consisting of MLXs 1-163;
(b) a polypeptide that comprises the amino acid sequence encoded by a polynucleotide selected from the group consisting of MLXs 1-163, in which one or more amino acids are substituted, deleted, inserted, and/or added and that has a biological activity equivalent to a polypeptide consisting of the amino acid sequence encoded by the polynucleotide; and
(c) a polypeptide encoded by a polynucleotide that hybridizes under stringent conditions to a polynucleotide selected from the group consisting of MLXs 1-163, wherein the polypeptide has a biological activity equivalent to a polypeptide consisting of the amino acid sequence encoded by the polynucleotide selected from the group consisting of MLXs 1-163;
(2) detecting the biological activity of the polypeptide of step (a); and
(3) selecting a compound that suppresses the biological activity of the polypeptide in comparison with the biological activity detected in the absence of the test compound.

12. A method of screening for a compound for treating colorectal cancer or preventing metastasis of colorectal cancer, said method comprising the steps of:
(1) contacting a test compound with a cell expressing one or more marker genes, wherein the marker genes are selected from the group consisting of MLXs 1-163; and
(2) selecting a compound that reduces the expression level of one or more of the marker genes.

13. The method of claim 12, wherein said cell expressing one or more marker genes comprises a colorectal cancer cell.

14. A method of screening for a compound for treating colorectal cancer or preventing metastasis of colorectal cancer, said method comprising the steps of:
(1) constructing a vector comprising the transcriptional regulatory region of a gene selected from the group consisting of MLXs 1-163 upstream of a reporter gene;
(2) transforming a cell with the vector of step (1);
(3) contacting a test compound with the cell of step (2);
(4) detecting the expression of the reporter gene; and
(5) selecting the test compound that suppresses the expression of the reporter gene compared to that in the absence of the test compound.

15. A kit comprising one or more detection reagents that respectively binds to one or more nucleic acid sequences selected from the group consisting of MLXs 1-163.

16. An array comprising one or more nucleic acids that respectively binds to one or more nucleic acid sequences selected from the group consisting of MLXs 1-163.

17. A method for treating colorectal cancer or preventing metastasis of colorectal cancer, said method comprising the step of administering a pharmaceutically effective amount of a compound that is obtained by the method according to any one of claims 10-14.

18. A method for treating colorectal cancer or preventing metastasis of colorectal cancer in a subject, said method comprising the step of administering to the subject a pharmaceutically effective amount of an antisense nucleic acids or small interference RNA against one or more genes selected from the group consisting of MLXs 1-163.

19. A method for treating colorectal cancer or preventing metastasis of colorectal cancer in a subject, said method comprising the step of administering to the subject a pharmaceutically effective amount of an antibody or fragment thereof that binds to a protein encoded by a gene selected from the group consisting of MLXs 1-163.

20. A method for treating colorectal cancer or preventing metastasis of colorectal cancer in a subject, said method comprising the step of administering to the subject a pharmaceutically effective amount of a polypeptide selected from the group consisting of (a)-(c), or a polynucleotide encoding the polypeptide or a vector comprising the polynucleotide:
(a) a polypeptide comprising the amino acid sequence encoded by a polynucleotide selected from the group consisting of MLXs 1-163 or fragment thereof;
(b) a polypeptide that comprises the amino acid sequence encoded by a polynucleotide selected from the group consisting of MLXs 1-163, in which one or more amino acids are substituted, deleted, inserted, and/or added and that has a biological activity equivalent to a protein consisting of the amino acid sequence encoded by the polynucleotide or fragment thereof; and
(c) a polypeptide encoded by a polynucleotide that hybridizes under stringent conditions to a polynucleotide selected from the group consisting of MLXs 1-163, wherein the polypeptide has a biological activity equivalent to a polypeptide consisting of the amino acid sequence encoded by the polynucleotide selected from the group consisting of MLXs 1-163 or fragment thereof.

21. A method for inducing an anti-tumor immunity, said method comprising the step of contacting with an antigen presenting cell a polypeptide selected from the group consisting of (a)-(c), or a polynucleotide encoding the polypeptide or a vector comprising the polynucleotide:
(a) a polypeptide comprising the amino acid sequence encoded by a polynucleotide selected from the group consisting of MLXs 1-163 or fragment thereof;
(b) a polypeptide that comprises the amino acid sequence encoded by a polynucleotide selected from the group consisting of MLXs 1-163, in which one or more amino acids are substituted, deleted, inserted, and/or added and that has a biological activity equivalent to a protein consisting of the amino acid sequence encoded by the polynucleotide or fragment thereof; and
(c) a polypeptide encoded by a polynucleotide that hybridizes under stringent conditions to a polynucleotide selected from the group consisting of MLXs 1-163, wherein the polypeptide has a biological activity equivalent to a polypeptide consisting of the amino acid sequence encoded by the polynucleotide selected from the group consisting of MLXs 1-163 or fragment thereof.

22. The method for inducing an anti-tumor immunity of claim 21, wherein the method further comprises the step of administering the antigen presenting cell to a subject.

23. A composition for treating colorectal cancer or preventing metastasis of colorectal cancer in a subject, said composition comprising a pharmaceutically effective amount of a compound that is obtained by the method according to any one of claims 10-14.

24. A composition for treating colorectal cancer or preventing metastasis of colorectal cancer in a subject, said composition comprising a pharmaceutically effective amount of an antisense nucleic acids or small interference RNA against one or more genes selected from the group consisting of MLXs 1-163.

25. A composition for treating colorectal cancer or preventing metastasis of colorectal cancer in a subject, said composition comprising a pharmaceutically effective amount of an antibody or fragment thereof that binds to a protein encoded by a gene selected from the group consisting of MLXs 1-163.

26. A composition for treating colorectal cancer or preventing metastasis of colorectal cancer in a subject, said composition comprising a pharmaceutically effective amount of a polypeptide selected from the group consisting of (a)-(c), or a polynucleotide encoding the polypeptide or a vector comprising the polynucleotide:
(a) a polypeptide comprising the amino acid sequence encoded by a polynucleotide selected from the group consisting of MLXs 1-163 or fragment thereof;
(b) a polypeptide that comprises the amino acid sequence encoded by a polynucleotide selected from the group consisting of MLXs 1-163, in which one or more amino acids are substituted, deleted, inserted, and/or added and that has a biological activity equivalent to a protein consisting of the amino acid sequence encoded by the polynucleotide or fragment thereof; and
(c) a polypeptide encoded by a polynucleotide that hybridizes under stringent conditions to a polynucleotide selected from the group consisting of MLXs 1-163, wherein the polypeptide has a biological activity equivalent to a polypeptide consisting of the amino acid sequence encoded by the polynucleotide selected from the group consisting of MLXs 1-163 or fragment thereof.
